(19) United States
(12) Patent Application Publication
(10) Pub. No.: US 2005/0227370 A1
(43) Pub. Date: Oct. 13, 2005

(54) Body fluid analyte meter & cartridge system for performing combined general chemical and specific binding assays

(76) Inventors: Urs A. Ramel, Sunnyvale, CA (US); Dillan Tay, San Jose, CA (US); Carole R. Stivers, Palo Alto, CA (US); Joel M. Blatt, Mountain View, CA (US); Benjamin R. Irvin, Cupertino, CA (US)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 11/038,213

(22) Filed: Jan. 21, 2005

Related U.S. Application Data
(60) Provisional application No. 60/551,595, filed on Mar. 8, 2004.

(51) Int. Cl. C12M 1/34; G01N 33/558; G01N 33/543

(52) U.S. Cl. 436/514; 435/287.2

(57) ABSTRACT

A combination body fluid analyte meter and cartridge system, having: (a) a body fluid analyte meter, with: a housing; a logic circuit disposed within the housing; a visual display disposed on the housing; and a measurement system disposed within the housing; and (b) a cartridge, having: at least one lateral flow assay test strip, the lateral flow assay test strip having: (i) a lateral flow transport matrix; (ii) a specific binding assay zone on the transport matrix for receiving a fluid sample and performing a specific binding assay to produce a detectable response, and (iii) a general chemical assay zone on the transport matrix for receiving the fluid sample and performing a general chemical assay to produce a detectable response; wherein the cartridge is dimensioned to be receivable into the body fluid analyte meter such that the measurement system is positioned to detect the responses in the specific binding assay zone and the general chemical assay zone in the lateral flow assay test strip.

### RELATED APPLICATIONS

The present application claims priority to U.S. Provisional Patent Application Ser. No. 60/551,595, filed Mar. 8, 2004, entitled Multi-Use Body Fluid Analyte Meter and Associated Cartridges, the entire disclosure of which is incorporated herein by reference in its entirety for all purposes.

### TECHNICAL FIELD

The present invention relates to body fluid analyte metering systems in general and, in one exemplary embodiment, to hemoglobin A1c (HbA1c) metering systems.

### BACKGROUND OF THE INVENTION

For many analytes such as the markers for pregnancy and ovulation, qualitative or semi-quantitative tests are appropriate. There are, however, a variety of analytes that require accurate quantitation. These include glucose, cholesterol, HDL cholesterol, triglyceride, a variety of therapeutic drugs such as theophylline, vitamin levels, and other health indicators. Generally, their quantitation has been achieved through the use of an instrument. Although suitable for clinical analysis, these methods are generally undesirable for point-of-care testing in physicians' offices and in the home due to the expense of the instrument.

The so-called “quantitative” analytical assays in the prior art do not in fact yield a true quantitative result. For example, U.S. Pat. No. 5,073,484 to Swanson discloses the “quantitative determination of an analyte” by using a cascade of multiple threshold test zones. Each test zone indicates in a binary manner that the amount of an analyte in a sample is either above or below a certain predetermined concentration. Each test zone thus determines only a comparison relative to a threshold value, and not an exact analyte concentration. Between successive test zones, only a range for the analyte concentration can be determined. Even comparing the results of each of the test zones, one cannot determine the exact analyte concentration. A true quantitative assay is not disclosed. Furthermore, the calibration curve of the Swanson assay is discontinuous, identifying discrete data points with no interpolation therebetween.

Another specific analyte that requires accurate quantitation is hemoglobin A1c (HbA1c), a form of glycated hemoglobin that indicates a patient's blood sugar control over the preceding two to three-month period. HbA1c is formed when glucose in the blood combines irreversibly with hemoglobin to form stable glycated hemoglobin. Since the normal life span of red blood cells is 90 to 120 days, the HbA1c will only be eliminated when the red blood cells are replaced. HbA1c values are thus directly proportional to the concentration of glucose in the blood over the full life span of the red blood cells and are not subject to the fluctuations that are seen with daily blood glucose monitoring.

The American Diabetes Association (ADA) recommends HbA1c as the best test to find out if a patient's blood sugar is under control over time. Performance of the test is recommended every three months for insulin-treated patients, during treatment changes, or when blood glucose is elevated. For stable patients on oral agents, the recommended frequency is at least twice per year.

While the HbA1c value is an index of mean blood glucose over the preceding two to three-month period, it is weighted to the most recent glucose values. This bias is due to the body's natural destruction and replacement of red blood cells. Because red blood cells are constantly being destroyed and replaced, it does not require 120 days to detect a clinically meaningful change in HbA1c following a significant change in mean blood glucose. Accordingly, about 50% of the HbA1c value represents the mean glucose concentration over the immediate past 30 days, about 25% of the HbA1c value represents the mean glucose concentration over the preceding 60 days and the remaining 25% of the HbA1c value represents the mean glucose concentration over the preceding 90 days.

The National Glycohemoglobin Standardization Program (NGSP) certifies laboratories and testing procedures for HbA1c, as well as establishes a precision protocol and other standardized programs. Recent studies have emphasized the clinical and therapeutic value of having HbA1c results immediately in the context of a physician office visit. Currently, patients needing to test for HbA1c must submit blood samples for laboratory analysis. The length of time that both the patient and medical professional have to wait is dependent on the availability of the laboratory resources. The patient's potential treatment is delayed pending the results of the test. This becomes a time-consuming and expensive treatment procedure that has diminished effectiveness.

The need for a truly quantitative and timely diagnostic assay, usable at the point-of-care, has recently taken on greater importance as numerous healthcare organizations have espoused disease management. One of the methodologies now being used to rationalize the use of disease management and demonstrate its return on investment is clinical risk stratification. This involves identifying and analyzing populations and sub-populations of patients with similar conditions and varying degrees of severity in the illness from which they suffer, and assessing their risk of experiencing certain adverse outcomes. Risk stratification provides the ability to segment a population into similar groups and subgroups, based on such factors (among others) as their relative risk of: suffering specific adverse outcomes (e.g. heart attacks, strokes, cancer, diabetic pregnancy, etc.); requiring hospitalization, emergency room, or physician office visitation; incurring certain levels of expenditure for diagnosis and treatment; and, mortality, morbidity, and other complications. When an organization has stratified patients according to their different levels of clinical risk, it can then design, develop and implement specific interventions that have a much greater chance of improving patient outcomes cost-effectively.

Thus, a need exists in the field of diagnostics for a method and device for accurate quantitation of analytes such as HbA1c which is sufficiently inexpensive, timely, efficient, durable, and reliable for use in a diagnostic device that would then permit point-of-care use by both trained and untrained individuals in locations such as the home, sites of medical emergencies, medical professional offices, and other locations outside of a clinic. Whether the device is disposable or reusable, fulfilling this need requires performing simultaneous, multiple assays from a single sample source.

### SUMMARY OF THE PRESENT INVENTION

In a first preferred embodiment, the present invention provides a combination body fluid analyte meter and cartridge system, including: (a) a body fluid analyte meter and (b) a cartridge having at least one lateral flow assay test strip therein, the lateral flow assay test strip having: (i) a lateral flow transport matrix; (ii) a specific binding assay zone on the transport matrix for receiving a fluid sample and performing a specific binding assay to produce a detectable response, and (iii) a general chemical assay zone on the transport matrix for receiving the fluid sample and performing a general chemical assay to produce a detectable response; wherein the cartridge is dimensioned to be receivable into the body fluid analyte meter such that a measurement system is positioned to detect the responses in the specific binding assay zone and the general chemical assay zone in the lateral flow assay test strip. Preferably, the measurement system is an optical measurement system. Most preferably, the measurement system is a reflectance measuring optical system.

In a second preferred embodiment, the present invention provides a cartridge for use with a body fluid analyte meter, the cartridge having at least one lateral flow assay test strip therein, the lateral flow assay test strip having: (i) a lateral flow transport matrix; (ii) a specific binding assay zone on the transport matrix for receiving a fluid sample and performing a specific binding assay to produce a detectable response, and (iii) a general chemical assay zone on the transport matrix for receiving the fluid sample and performing a general chemical assay to produce a detectable response; wherein the cartridge is dimensioned to be receivable into a body fluid analyte meter such that a measurement system in the body fluid analyte meter is positioned to detect the responses in the specific binding assay zone and the general chemical assay zone in the lateral flow assay test strip.

In a third preferred embodiment, the present invention provides a lateral flow assay test strip, having: (i) a transport matrix; (ii) a specific binding assay zone on the transport matrix for receiving a fluid sample and performing a specific binding assay to produce a detectable response, and (iii) a general chemical assay zone on the transport matrix for receiving the fluid sample and performing a general chemical assay to produce a detectable response, wherein the lateral flow assay test strip is formed from a single continuous membrane of material.

In a fourth preferred embodiment, the present invention provides a transverse flow assay test strip, having: a transport matrix comprising a stack of membranes; a specific binding assay zone on the transport matrix for receiving a fluid sample and performing a specific binding assay to produce a detectable response, and a general chemical assay zone on the transport matrix for receiving the fluid sample and performing a general chemical assay to produce a detectable response.

In a fifth preferred embodiment, the present invention provides a lateral flow assay test strip, having: a lateral flow transport matrix; a specific binding assay zone on the transport matrix for receiving a fluid sample and performing a specific binding assay to detect the level of human albumin present in the fluid sample, and a general chemical assay zone on the transport matrix for receiving the fluid sample and performing a general chemical assay to detect the level of creatinine present in the fluid sample.

### OPERATION AND ADVANTAGES OF THE PRESENT INVENTION

In its various aspects, the present invention provides a system and method for performing a specific binding assay and a general chemistry assay together in a lateral flow assay format, thus determining quantitatively the level of one or more analytes from a single sample source.

Optionally, the measurement of one analyte can be used to obtain or correct the measurement of another analyte in the same sample. In particular examples, a system is provided for quantitatively determining the amount of glycated hemoglobin (HbA1c) by detecting the level of HbA1c using a specific binding assay and detecting the level of total hemoglobin (Hb) present in the sample using a general chemistry assay.

The present invention provides a system for determining the level of a plurality of analytes in a sample. This system preferably includes at least one test strip having a transport matrix configured for moving the sample in a lateral flow thereacross. The present invention may optionally be self-contained (e.g.: in a single-use disposable device) or may comprise a re-usable meter with a series of disposable cartridges that contain one or more of the transport matrices.

Each transport matrix preferably includes a specific binding assay zone for receiving the sample and performing a specific binding assay to produce a detectable response. Each transport matrix also preferably includes a general chemical assay zone for receiving the sample and performing a general chemical assay to produce a detectable response directly or through a chemical modification. The present invention also includes systems for determining the analyte levels in the sample from the detectable responses in the specific binding assay and general chemical assay zones.

The present invention also provides a system for determining the level of a first and a second analyte in a sample that contains a chemical indicator for chemically reacting with the second analyte to produce a detectable result. The system includes one or more transport matrices for moving the sample in a lateral flow thereacross. Each transport matrix preferably includes a conjugate zone that receives and contacts the sample with a labeled indicator reagent diffusively immobilized thereon. The labeled indicator reagent reacts in the presence of the first analyte to form a mixture containing a first analyte:labeled indicator complex. Each transport matrix preferably includes a capture zone (i.e.: the specific binding assay zone) that receives and contacts the mixture from the conjugate zone with a first reagent non-diffusely immobilized on the transport matrix. The first reagent reacts in the presence of the mixture to form a detectable response from the level of the labeled indicator reagent immobilized in the capture zone and a detectable response from the level of the second analyte present in the mixture in the capture zone. In particular embodiments of the invention, the transport matrix optionally further includes an interference removal (conjugate removal) zone that receives and immobilizes the first analyte:labeled indicator reagent complex from the remaining mixture. A measurement zone (i.e.: the general chemical assay zone) on each transport matrix receives the remaining mixture from the interference removal zone and measures the detectable response from the reaction between a chemical indicator and the second analyte. Alternatively, the labeled indicator reagent and the first analyte:labeled indicator complex are simply washed past a measurement zone to a capture zone. In such embodiments, the analyte:labeled indicator complex may be further washed into a terminal absorbent pad. The present invention preferably includes systems for determining the levels of the first and second analytes in the sample from the detectable responses in the capture zone and measurement zone. As will be shown, such systems may comprise optical (e.g.: reflectance measuring) detectors. It is to be understood, however, that the present invention is not so limited. For example, other optical as well as non-optical measurement/detection systems may also be used for detecting the specific binding assay and general chemical assay responses, all keeping within the scope of the present invention.

The present invention also provides either a single-use assay metering device, or a multi-use meter with single-use cartridges receivable therein, for analyzing a plurality of analytes. The single-use embodiments preferably include a unitary housing having an exterior surface and sealing an interior area and a sample receptor that receives a sample containing a plurality of analytes selected for determining their presence. The sample receptor is located on the exterior surface of the housing. In optional embodiments, both the single-use meter system and the multi-use meter and single-use cartridge system also includes a sample treatment system that reacts the sample with a self-contained reagent to yield a physically detectable change that correlates with the amount of one of the selected analytes in the sample. Such sample treatment system may optionally be sealed within the housing and in fluid communication with the sample receptor or may be contained in a sample receptacle that is external to the instrument (and its cartridge). The present invention further includes detectors that respond to the physically detectable change in a plurality of detection zones and produce an electrical signal that correlates to the amount of the selected analyte in the sample. Such detectors are sealed within the housing of the meter. The present invention also includes a processor that stores assay calibration information uniquely characteristic for determining the level of a first and second analyte in the sample from the detectable responses in the specific binding assay and general chemical assay detection zones. The processor further calibrates the detectors using stored detector calibration information and converts the electrical signal to a digital output that displays the assay results. The processor is sealed within the housing and is connected to the detectors. The present invention also includes an output device that delivers the digital output external to the housing. The output device is connected to the processor.

In the embodiment of the invention in which disposable cartridges are used, such single-use cartridges optionally include a unitary housing having an exterior surface and sealing an interior area and a sample receptor that receives a sample containing a plurality of analytes selected for determining their presence. The sample receptor is located on the exterior surface of the cartridge housing. The cartridge also includes the sample treatment system that reacts the sample with a self-contained reagent to yield a physically detectable change that correlates with the amount of one of the selected analytes in the sample. The sample treatment system is sealed within the cartridge housing and in fluid communication with the sample receptor or may be contained in a sample receptacle external to the instrument and cartridge.

In the embodiment of the invention in which a multi-use meter is used, the multi-use meter includes the detectors that respond to the physically detectable change in a plurality of detection zones and produces an electrical signal that correlates to the amount of the selected analyte in the sample. The detectors are sealed within the meter housing. The meter includes the processor that stores assay calibration information uniquely characteristic to the set of single-use cartridges supplied with the meter for determining the level of a first and second analyte in the sample from the detectable responses in the specific binding assay and general chemical assay detection zone. The processor further calibrates the detector using stored detector calibration information and converts the electrical signal to a digital output that displays the assay results. The processor is sealed within the instrument housing and is connected to the detectors. The meter also includes an output device that delivers the digital output external to the housing. The output device is connected to the processor.

A diagnostic kit is included in the present invention for determining the levels of a first and a second analyte in a sample. The kit includes a sample receptacle containing a chemical indicator for performing a general chemical assay on the sample, by reacting with the second analyte to produce a detectable result, and a single-use meter or a multi-use meter and disposable cartridge as recited above.

A transport matrix for determining the level of a plurality of analytes in a sample is included in the present invention. In one embodiment, the transport matrix includes at least one membrane for moving the sample in a lateral flow theracross. A specific binding assay zone on the membrane receives the sample and performs a specific binding assay to produce a detectable response and a general chemical assay zone on the membrane receives the sample and performs a general chemical assay to produce a detectable response directly or through a chemical modification. In various configurations, the general chemical assay zone may be located either upstream or downstream from the specific binding assay zone.

The present transport matrix is used for determining the level of a first and a second analyte in a sample. The sample contains a chemical indicator for chemically reacting with the second analyte to produce a detectable result. The transport matrix optionally includes at least one membrane for moving the sample in a lateral flow across the transport matrix. The membrane includes a conjugate zone that receives and contacts the sample with a labeled indicator reagent diffusively immobilized on the membrane. The labeled indicator reagent reacts in the presence of the first analyte to form a mixture containing a labeled first analyte:indicator complex. The membrane also includes a capture zone (i.e.: the specific binding assay zone) that receives and contacts the mixture from the conjugate zone with a first reagent non-diffusely immobilized on the membrane in the capture zone.

Preferably, the first reagent reacts in the presence of the mixture to form a detectable response from the level of the labeled indicator immobilized in the capture zone and a detectable response from the level of the second analyte present in the mixture in the capture zone. An optional interference removal (conjugate removal) zone on the membrane receives and immobilizes the first analyte:labeled indicator complex as well as any uncomplexed labeled indicator reagent from the remaining mixture. In one preferred configuration, a measurement zone (i.e.: the general chemical assay zone) on the membrane receives the remaining mixture from the interference removal zone and measures the detectable response from reacting the chemical indicator and the second analyte. In another preferred configuration, the measurement (i.e.: general chemical assay) zone is upstream from the capture (i.e.: specific binding) zone and the labeled indicator reagent and the first analyte:labeled indicator complex are washed past the measurement zone to a capture zone. In this second preferred configuration, the analyte:labeled indicator complex is further washed into a terminal absorbent pad.

Instead of the preferred competitive inhibition specific binding assay described above, the transport matrix can alternately provide a specific binding assay that is a direct competitive assay or a sandwich assay. Various alternate embodiments of the inventive transport matrix include reversing the sequence of the specific binding and general chemical assay zones for performing the specific binding assay and general chemical assay as well as increasing the total number of zones present on the transport matrix.

The present invention also provides a method for determining the presence of at least a first and second analyte from a plurality of analytes in a sample using different types of assays on the same sample, the method comprising the steps of: treating the sample with a chemical indicator for chemically reacting with or modifying the second analyte to produce a detectable result from a general chemical assay; treating the same sample portion with a labeled indicator reagent to create a conjugate with the first analyte, or to compete with the analyte for binding to a specific binding partner, to produce a detectable result from a specific binding assay; transporting the sample sequentially across the plurality of zones for detecting a response from the first analyte conjugate in one zone and detecting a response from the chemical indicator second analyte in a second zone; and determining the analyte levels in the sample from the detectable responses in the first and second zones.

The present invention includes another method for determining the level of at least two analytes in a sample. The method includes the steps of: contacting the sample with an end portion of a transport matrix having a plurality of zones; transporting the sample to a labeled indicator reagent diffusively immobilized on the transport matrix; reacting the labeled indicator reagent in the presence of a first analyte to form a mixture; transporting the mixture to a first reagent non-diffusely immobilized on the transport matrix; reacting the first reagent in the presence of the mixture to form an immobilized first reaction product and a detectable response related to one or more of the analyte levels in the sample; transporting the remaining mixture without the labeled indicator to a second reagent non-diffusely immobilized on the transport matrix; reacting a chemical indicator with the remaining sample to form a second reaction product and a detectable response related to the second analyte level in the sample; determining one or more of the analyte levels in the sample from the detectable responses in the reacting steps with the first and second reagents.

Another method included in the present invention determines the level of one or more analytes in a sample using the steps of: moving a sample in a lateral flow across a transport matrix; performing a specific binding assay on the sample in a specific binding assay zone on the transport matrix to produce a detectable response; performing a general chemical assay on the sample in a general chemical assay zone on the transport matrix to produce a detectable response; and determining the levels of one or more analytes in the sample from the detectable responses in the specific binding assay and general chemical assay zones. Alternatively, the sequence of specific binding and general chemical assays may be reversed.

In preferred embodiments, the present meter measures hemoglobin A1c (HbA1c), but is not so limited. In various preferred aspects of the present invention, a drop of blood to be analyzed is placed into the disposable cartridge, with the cartridge being received into the meter.

Another advantage provided by the present invention is the ability to produce quantitative results in a single step—requiring only sample introduction into the device to activate its functioning. A digital result is produced within minutes from either a treated or an untreated sample. Electronics, detector systems (e.g., reflectance measurement systems), a high resolution analog-to-digital signal converter, integrated temperature measurement systems (to provide automatic temperature correction, if needed), a digital display for unambiguous readout of analyte result(s), and an electronic communications port for transfer of results to a computer or laboratory or hospital information system may all be contained within the present invention. Other systems for communication of the assay result(s) may be utilized, including but not limited to acoustic or audible means (including spoken words) and tactile means (including Braille).

The present invention, in some of its preferred embodiments, avoids the limitations of prior art systems that required a sample treatment, or pretreatment, of some type before the sample is applied to the assay device. Examples of sample treatments that might otherwise have to be performed outside of the assay device are blood separation (to produce plasma), accurate and precise volume measurement, removal of interfering materials (chemical interferents, sediments), dilution, etc. Alternately, the sample can be extracted from another device that provides sample treatment. Such treatments are not precluded by the present invention, and may include the use of specialized sample treatment devices. Examples of such devices include, but are not limited to, dilution devices where a small volume of blood is diluted and/or lysed and blood sampling and/or separation devices where a small volume of plasma may be produced. Such devices may be entirely separate from or attached (permanently or temporarily) to the present invention.

An example of a treatment specific to the measurement of HbA1c is dilution into a solution containing sodium ferricyanide, surfactant and a pH buffer, including optionally additional salts, proteins or other polymeric substances to improve assay performance or resistance to interfering substances. The diluent solution may be contained in a small screw cap vial (preferably under 2 mL in volume) and supplied as part of an assay kit that may also include a capillary device for obtaining a small sample of whole blood (preferably 10 μL or less) from a finger stick. This capillary may then be used to transfer the blood sample into the diluent. After mixing, a transfer pipette or dropper may be used to place the diluted sample into the sample port of the present invention.

The present multi-use meter and disposable cartridge embodiments of the present invention offer numerous advantages, including, but not limited to, the following.

First, although the cartridges are disposable, the meter itself can be used again and again. Thus, many of the more expensive components of the system, including the logic circuit, the electronics and the optical measurement system can be incorporated into the meter. As such, these components need not be discarded after every use. This results in cost savings to the manufacturer and to the user.

A second advantage of the present cartridges is that they avoid the use of a desiccant within the meter itself. This is due to the fact that the sensitive test strips are positioned within each of the individual cartridges. Since such individual cartridge can be enclosed in moisture proof wrapping (which may be removed immediately before use), the test strips therein can be kept dry without the need for a desiccant in the meter housing. The removal of the desiccant from the present meter results in space savings, producing a compact, reduced cost, device.

A third advantage of the present cartridge system is that the actual blood sample to be analyzed does not contaminate the inner workings of the (multi-use) meter. Rather, the blood sample is at all times contained within the (disposable) cartridge itself. The advantage of this system is that it instead simply presents the analysis of the blood sample in a format to be read by an optical system in the meter, without having to decontaminate or dispose of the meter.

A fourth advantage of the present cartridge system is that, in embodiments where the cartridges and meter are matched to each other, no calibration information need be presented by the disposable cartridge to the meter, thus saving cost.

### DEFINITIONS AND AN EXPLANATION OF ACCURACY, SENSITIVITY AND RESOLUTION AS DESCRIBED HEREIN

As stated above, the present invention provides a novel and unobvious assay device and method for quantifiably identifying multiple analytes using both a specific binding assay and general chemical assay on the same sample at the same time. The quantification obtained by the present invention can be defined by measures including assay accuracy, sensitivity, and resolution.

The term, body fluid analyte, is taken to mean any substance of analytical interest, including, but not limited to, hemoglobin A1c, cholesterol, triglycerides, albumin, creatinine, human chorionic gonaotropin (hCG), or the like, in any body fluid, such as blood, urine, sweat, tears, or the like, as well as fluid extracts of body tissues, whether applied directly to the present invention or as a diluted solution.

As defined herein, sensitivity is the lower detection limit of an assay or clinical chemistry. The lower detection limit is the lowest detectable amount of analyte that can be distinguished from a zero amount, or the complete absence, of an analyte in a sample. The lowest detectable amount of analyte is preferably calculated from a calibration curve that plots the assay signal versus analyte concentration. The standard deviation of the mean signal for a zero calibrator is determined first. Twice the standard deviation is then added to or subtracted from the mean signal value as the case may be. Subsequently, the analyte concentration that is directly read from or calculated from the calibration curve is the lower detection limit.

It should be understood that the present invention is not limited to any one method of determining sensitivity, or any other quantitative measurement systems. For example, an alternative method that can be used is to determine the mean and standard deviation of several calibrators, including zero. The lowest concentration that is distinguishable from the zero calibrator is experimentally determined with an acceptable degree of statistical confidence, e.g. 95% or greater. A variation on this approach is to determine the lowest concentration of analyte that can be measured with a given level of imprecision, e.g. 15% or less. This analyte concentration value is often called the limit of quantitation.

Another method of determining the sensitivity of an assay uses an analytical chemistry approach to refer to the slope of the curve comparing the assay signal to the analyte concentration. The greater the absolute value of the slope of the curve, the greater the sensitivity. For example, using reflectance as the method of measuring the physical detectable change as demonstrated by the test results provided herein, a curve exhibiting greater reflectance change per unit change in analyte concentration would be more sensitive. However, the assay signal versus analyte concentration curve is usually nonlinear. As a result, the curve has regions that are more or less sensitive, directly affecting the usefulness of the assay results. Another problem is that this method of determining sensitivity does not take into account whether a given signal change is significant as compared to the level of noise in the measurement system.

Resolution, as used herein, is defined as the ability of the test to distinguish between closely adjacent, but not identical, concentrations of analyte as a function of total imprecision (total CV) in the way that sensitivity (the lower detection limit) is defined. The lower the overall noise or imprecision of the test (the lower the CV), the greater the resolving power or resolution. The individual components of resolution include analog to digital conversion resolution (the number of bits available to create a digitally-encoded number from the analog signal), noise in the analog part of the instrument measurement system, and noise inherent in the chemistry system (including flow irregularities, material variability, assembly variability, and formulation variability).

Accuracy, as defined herein, is the ability of the assay to yield a result that correlates closely with the result from a reference or predicate assay. Specifically, accuracy is defined in terms of mean bias from a reference. The bias is the difference between the experimental and reference values. If the bias is zero (i.e., they are identical), then the test is 100% accurate. In order to distinguish error due to imprecision from error due to inaccuracy or bias, mean values from a series of replicate determinations are used. Of course, this definition presumes that the predicate assay yields a true value.

The accuracy of the inventive assay is further improved by supplying the microprocessor of the assay device with exact parameter values and equations for calibration as well as the exact parameter values to correct for variations in LED spectral output. These exact calibration parameters and equations are loaded electronically into the assay device (i.e.: the meter or the cartridges, or both) during manufacture of the present invention. This inventive method eliminates another source of error by avoiding the prior art's reliance on a series of discrete pre-programmed constants or equations built into a reusable instrument.

The present invention improves the assay's accuracy by correcting for errors that can occur at several levels. For example, the present invention preferably uses an assay that advantageously decreases the mean bias by factory-calibration against standard materials and laboratory reference methods. The inventive method avoids the use of simultaneous on-board reference assays disclosed in the prior art that introduce a background error for the reference test that cannot be corrected. It also avoids the errors inherent in the use of secondary standard materials by a user who must calibrate an instrument periodically in a clinical laboratory.

Another example is the preferred use by the present invention of clinical samples for calibration. By calibrating with clinical samples, or synthetic calibrators if they yield the same values as clinical samples, the issue of errors caused by clinical background or matrix effects is minimized.

Another example is that measurement background or error can arise from within the measurement system. It includes transport matrix alignment errors (in all three dimensions), LED spectral variability (calibrated during manufacture), LED energy emission variability, optical alignment variability, and variability in the amplification and measurement of the analog electrical signals arising from the detectors. Virtually all of these effects can be eliminated by using a ratiometric strategy—ratioing the detector output signals to the detector signals obtained from the initial dry strip readings and to the output from the reference detector.

The ratiometric strategy of reflectance measurement is illustrated in Equation 1 below. This strategy provides for internal cancellation of most gain (slope, or proportional) and offset (intercept, or fixed value) errors that will occur in both the optics (or other detector systems) and electronics, and is used for all analyses. Use of Equation 1 reduces reflectance variability by about 10-fold. In this equation, “R” is reflectance. Initial readings are taken on the dry strip and then all subsequent readings are ratioed to that initial value after subtraction of blank (dark current, “OFF”) readings. All readings are ratioed to the signal at the reference photodetector (“ref”), also after subtraction of a blank (dark current) reading. Equation 1 reads as:

$$R = \frac{( \frac{{R}_{final : ON} - {R}_{final : OFF}}{{ref}_{final : ON} - {ref}_{final : OFF}} )}{( \frac{{R}_{initial : ON} - {R}_{initial : OFF}}{{ref}_{initial : ON} - {ref}_{initial : OFF}} )}$$

Exemplary definitions of the functions of the transport matrix can include, for example and not for limitation:

Capture zones, wherein a detectable change is localized by specific binding in order to facilitate measurement, and an optimized capture zone provides a uniform distribution of detectable change;

Conjugate zones, where conjugates, antibodies, antigens, and the like are diffusively immobilized and where they first react with or encounter analyte in the sample fluid. An optimized conjugate zone produces a uniform mixture of conjugate and other diffusively immobilized materials with the sample fluid, and is preferably located as close to the capture zone as is compatible with an appropriately sensitive detectable response. The dissolution of these materials is preferably complete or substantially complete within the time period of the assay;

Non-specific or general chemistry measurement zones, where a detectable change, as in the case of an indicator or analyte having a detectable characteristic (such as absorption of light at a specific wavelength), is not specifically localized, but rather is distributed evenly throughout the material so as to present a representative portion of the sample to the detector(s) for measurement of concentration;

Interference removal zones, where substances in the sample fluid are removed or modified so that they no longer can alter the magnitude of detectable change in subsequent capture zones. An optimized interference removal zone is capable of removing or modifying an interfering substance or substances, up to a specified concentration, so that they exert either no bias or an acceptable bias on the analyte result;

Sample pretreatment zones, where the chemical composition of the sample is modified in order to make it more compatible with subsequent functional elements of the assay. A sample pretreatment zone, when optimized, adjusts other important chemical properties of the same, such as pH, ionic strength, and the like, so that they are appropriate for the proper functioning of the other chemical elements on the strip;

Blood separation zones, where red blood cells are removed from the sample fluid to produce plasma or similar uncolored fluid. A preferred blood separation zone will remove red blood cells and other cellular components of whole blood as needed, so that only an acceptable number of these components remain in the resulting plasma, and hemolysis is minimal. For instance, acceptable levels of hemolysis (release of free hemoglobin) in some assays may be defined by whether hemoglobin color is detectable by the detector(s) and can preferably mean a level of hemolysis that is nearly zero (<<1%) to about 2%;

Sample overflow areas provide for wide sample volume tolerance, wherein excess sample volume, beyond that required to perform the assay, is absorbed. A preferred sample overflow zone will accommodate sample volumes over the specified range without introducing bias in the analyte result within a specifically acceptable or tolerable range of error;

Sediment filtration zones, wherein particulate materials in the sample are removed to yield an optically clear fluid. A preferred sediment filtration zone will remove particulate materials that may interfere with uniform fluid flow or production of a detectable change to the extent that samples with sediment do not produce unacceptable bias in the reported analyte result;

Conjugate removal zones, wherein labeled indicator reagent and its complexes are removed in a manner similar to those described for interference removal and sediment filtration zones. A preferred conjugate removal zone will remove labeled indicator reagent and its complexes that may interfere with production of a detectable change, so that they do not exert any significant bias on the analytical result;
and others that may be unique to a variety of sample fluids or analytes (whole blood, plasma, serum, urine, saliva, vaginal swabs, throat swabs, mucous secretions from various parts of the body, sweat, digested tissue samples, etc.).

The preferred materials for these functions vary with the specific function required and may include:
for the sample pretreatment zone, detection zone, and other areas not specifically designated, nitrocellulose as described above;
for the non-specific measurement zones, uniform (symmetric or asymmetric) microporous filtration membranes such as nylon membranes produced by Pall Gelman and CUNO and polyethersulfone membrane produced by Pall Gelman, either unmodified or modified chemically to change the adsorption properties of the membrane so as to specifically adsorb an interferent or prevent adsorption of the analyte;
for the sediment filtration and blood separation zones treated glass fiber composites with a binder, mixed cellulose glass fiber composites with a binder, composites of polyester and glass fiber, “shark skin”-like materials, and microporous filtration membranes such as nylon membranes supplied by Pall Gelman, Millipore and CUNO as well as asymmetric polysulfone membrane produced by Memtec and Presence® polyethersulfone membrane produced by Pall Gelman;
for the conjugate zone open structure materials, such as polyester nonwoven composites, cellulose acetate membranes, and glass fiber materials with binder—alone or treated with conjugate-releasing materials (polyols, surfactants, hydrophilic polymers, copolymers, or the like);
for the interference removal and conjugate removal zones ion exchange materials, such as Whatman GF/QA, polymer membranes which contain diffusively immobilized interference removal materials such as heterophilic blockers, anti-HAMA (Human-Anti-Mouse-Antibodies) materials, and chaotropic agents, as well as treated glass fiber composites with a binder, mixed cellulose glass fiber composites with a binder, composites of polyester and glass fiber, “shark skin”-like materials, and microporous filtration membranes such as nylon membranes produced by Pall Gelman and CUNO as well as asymmetric polysulfone membrane produced by Memtec and Presence® polyethersulfone membrane produced by Pall Gelman; and
for sample overflow areas absorptive materials, such as Transorb® produced by Filtrona Richmond.

In one exemplary embodiment, a multi-segmented transport matrix specific to the measurement of HbA1c includes:
for conjugate zone material, cellulose acetate membrane;
for capture (specific binding) zone material, nitrocellulose membrane; and
for non-specific (general chemistry) measurement zone material, nylon. In this specific example of measurement of HbA1c, the material also serves as a conjugate removal zone that filters out particulate conjugate and prevents its color from interfering with the measurement of total hemoglobin. The filtration properties of this material may be dependent on, but are not limited to, membrane pore size, surface charge of the membrane and addition of chemicals that may create opportunities for chemical attraction or repulsion based on but not limited to ionic, dipole-dipole and hydrophobic interactions.

As will be shown herein, however, various embodiments of the present invention entail using the same material for more than one of the functions required of the transport matrix. For example, a nitrocellulose membrane may serve the functions of conjugate zone, capture (specific binding) zone, and non-specific (general chemistry) measurement zone. Alternately, nitrocellulose may serve the functions of capture (specific binding) zone and non-specific (general chemical assay) measurement zone and cellulose acetate may serve the function of the conjugate zone. In a further example, nitrocellulose serves the functions of the conjugate zone and capture (specific binding) zones, and nylon serves the function of a non-specific (general chemical assay) measurement zone.

General chemistry assays are defined to include reactions performed for analytes such as, but not limited to, glucose, creatinine, cholesterol, HDL cholesterol, LDL cholesterol, triglycerides, and urea nitrogen (BUN). For general chemistry assays, the present invention preferably uses enzyme-catalyzed reactions to produce a detectable response or signal in each detection zone related to a unique value for the level of analyte in the sample. Other systems for producing a detectable response in the detection zones are also suitable for use in the present invention. For example, and not for limitation, the analyte may react with an enzyme or sequence of enzymes to produce a detectable product by reduction, oxidation, change of pH, production of a gas, or production of a precipitate. Non-enzymatic reactions, whether catalyzed or not, may also take place either together with or in place of enzymatic reactions. Examples of detectable products include those which may be detected by fluorescence, luminescence, or by reflectance or absorbance of a characteristic light wavelength, including wavelengths in the ultraviolet, visible, near infra-red, and infrared portions of the spectrum. The term “indicator”, as used herein for general chemistry assays, is meant to include all compounds capable of reacting with the analyte, or an analyte reaction product that is stoichiometrically related to an analyte, and generating a detectable response or signal indicative of the level of analyte in the sample.

Specific binding assays are defined to include reactions between specific binding partners such as, but not limited to, lectin carbohydrate binding, complementary nucleic acid strand interactions, hormone receptor reactions, streptavidin biotin binding, and immunoassay reactions between antigens and antibodies. For specific binding assays, the present invention preferably uses particle detection for a detectable response or signal in each reaction zone related to the level of analyte in the sample. Other systems for providing a detectable response in the specific binding zones are suitable for use in the present invention. For example, and not for limitation, the analyte or its specific binding partner may be labeled either directly or indirectly by means of a second antibody conjugate or other binding reaction with an indicator to measure fluorescence or luminescence, or the reflectance or absorption of a characteristic light wavelength. As used herein for specific binding assays, “indicator” is meant to include all compounds capable of labeling the analyte or its specific binding agents or conjugates thereof and generating a detectable response or signal indicative of the level of analyte in the sample.

Although the chemistry and configurations of the present invention may be used in an integrated assay device, the present invention can be used in any other instrumented reflectance or transmission meter as a replaceable reagent. Thus, the present invention also encompasses integrated assay instruments and analytical assay instruments, including replaceable cartridges in a limited re-use analytical instrument, comprising the present assay device.

### BRIEF DESCRIPTION OF THE DRAWINGS

FIG. 1A is an exploded perspective view of a preferred embodiment of a single-use meter diagnostic device of the present invention;

FIG. 2A is a side view of one embodiment of an HbA1c dry reagent assay transport matrix schematically illustrating the functional elements involved in a specific binding assay and general chemical assay;

FIG. 2B is a top plan view of the transport matrix illustrated in FIG. 2A;

FIG. 2C is a side view of an alternative transport matrix employing a single membrane with a specific binding assay zone upstream of a general chemical assay zone;

FIG. 2D is a side view of an alternative transport matrix employing a single membrane with a specific binding assay zone downstream of a general chemical assay zone;

FIG. 2E is a side view of an alternative transport matrix employing a single membrane material with conjugate disposed between the specific binding assay zone and the general chemical assay zone;

FIG. 2F is a side view of an alternative transport matrix employing nitrocellulose and cellulose acetate membranes with the specific binding assay zone and the general chemical assay zone disposed on the nitrocellulose;

FIG. 2G is a side view of an alternative transport matrix, similar to FIG. 2F, but with the specific binding assay and general chemical assay zones reversed;

FIG. 2H is a side view of an alternative transport matrix having the conjugate zone and specific binding assay zone disposed on a first membrane and a general chemical assay zone disposed on a second membrane.

FIG. 2I is a side view of an alternative transport matrix employing a conjugate removal zone on a first membrane with a spreader layer under second membrane upon which the general chemistry assay zone is disposed;

FIG. 2J is a side view of an alternative transport matrix, similar to FIG. 21, but employing a conjugate pad;

FIG. 2K is a side view of an alternative transport matrix, similar to FIG. 21, but employing an additional layer forming a conjugate trap under the spreader layer;

FIG. 2L is a side view of an alternative transport matrix employing a spreader layer under a first membrane with a specific binding assay zone thereon. A general chemical assay zone is disposed on a second membrane.

FIG. 3A is an exploded side view of an alternative embodiment of the inventive transport matrix illustrating the functional elements involved in a specific binding assay and general chemical assay that employs transverse flow;

FIG. 3B is an exploded side view of an alternative embodiment of the inventive transport matrix that employs a combination of lateral and transverse flow;

FIG. 4 is a perspective view of an embodiment of the disposable cartridge and multi-use meter system of the present invention.

FIG. 5A is an exploded perspective view of an embodiment of the cartridge of the present invention.

FIG. 5B is a top plan view of the bottom of the single-use cartridge, showing the test strips received therein.

FIG. 5C is bottom plan view of the top of the single-use cartridge.

FIG. 5D is a top plan cut away view of the single-use cartridge received into the multi-use meter, showing the alignment of the test strips in the cartridge to the optical detectors in the meter.

FIG. 6 is an exploded perspective view of the multi-use meter.

FIG. 7 is a sample standard curve for analyte 2 showing concentration vs. reflectance;

FIG. 8 is a graph depicting an algorithm for determining the concentration of analyte 1 from reflectance readings in detection zone 1 and the concentration of analyte 2 as determined from detection zone 2 (general chemistry assay zone).

FIG. 9 is a graph of the linearity of recovery data for % HbA1c;

FIG. 10A is a graph of the effect of hematocrit on HbA1c test results for a low % HbA1c (non-diabetic) sample;

FIG. 10B is a graph of the effect of hematocrit on HbA1c test results for a high % HbA1c (diabetic) sample;

FIG. 11A is a graph of percent HbA1c correlation from finger stick samples obtained by professionally trained medical personnel; and

FIG. 11B is a graph of percent HbA1c correlation from finger stick samples obtained directly by users.

Like reference numerals refer to like elements throughout the attached drawings.

### DETAILED DESCRIPTION OF THE DRAWINGS

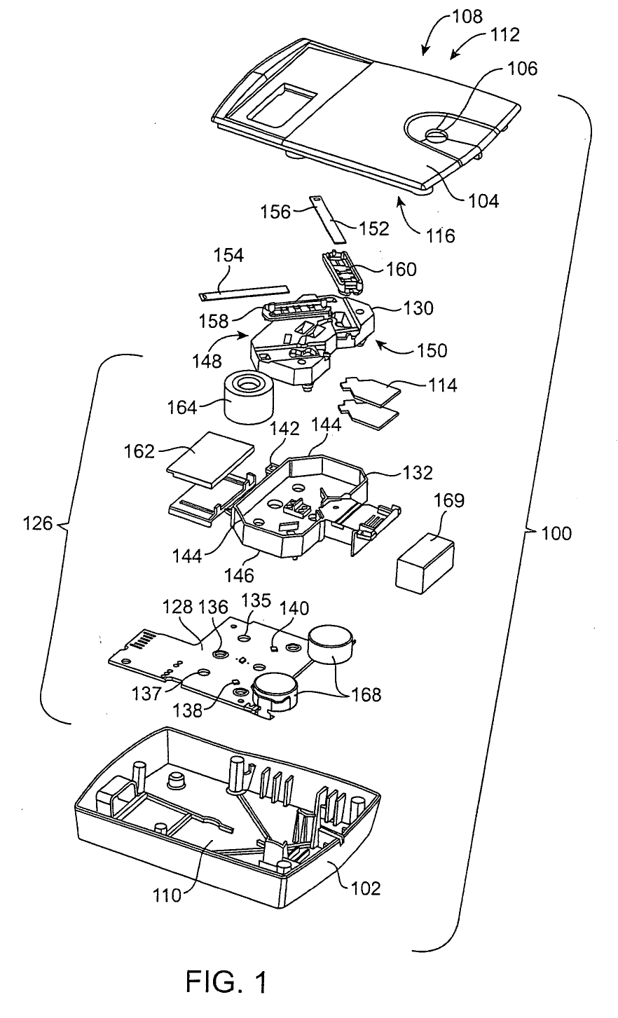

A preferred embodiment of a single-use meter diagnostic device 100 for measuring HbA1c is illustrated in FIG. 1. Meter 100 includes a housing 102 and cover 104 having a receptor such as inlet port 106 that extends from the exterior surface 108 of the cover to the interior 110 of the housing for receiving a sample 112 containing the one or more selected analytes to be determined.

The inlet port 106 allows the sample 112 to be introduced to a sample receiving device 114 which is attached to the interior surface 116 of the cover 104. The sample receiving device 114 includes a two-layer pad which is in fluid communication with two assay strips and serves to distribute the sample between the two strips. Optionally, the sample receiving device 114 can also include a sample filter pad which removes undesired contaminants from the sample. The sample filter pad can be the same as the receiving pad with one pad performing both functions. Meter 100 can include more than one sample filter pad along the pathway of the sample flow that remove different types of contaminants. The two assay strips contain chemical reagents for determining the presence of one or more selected analytes.

The interior 110 of the housing encloses a reflectometer 126 that includes a printed wiring assembly having a printed circuit board (PCB) 128. The reflectometer 126 also includes an optics assembly 130 and a shield 132. The PCB 128 has one face 134 with a reference detector 136 and zone detectors 138, 140 mounted directly thereto. The face 134 of the PCB also has two light-emitting diodes (LEDs) 135, 137, one for each pair of illumination channels, mounted directly to the PCB. The LEDs 135, 137 are preferably in bare die form without an integral lens, enclosure, or housing. As a result, the LEDs 135, 137 provide illumination in all directions above the face 134 and are directed only by the optics assembly 130. Similarly, the zone detectors 138, 140 and reference detector 136 are bare die mounted directly to the face 134 of the PCB. The LEDs 135, 137 and the detectors 136, 138, 140 are all positioned in the same plane.

FIG. 1 also illustrates the position of the shield 132 relative to the PCB 128. Aperture 142 is provided through the shield 132 to prevent obstructing the LEDs 135, 137 and the reference detector 136. Openings 144 are provided to prevent obstructing zone detectors 138, 140. The shield 132 includes upstanding walls 146 which prevent stray radiation from entering the zone detectors 138, 140. The upstanding walls 146 are positioned adjacent the reflecting and refracting elements of the optics assembly 130 when the reflectometer 126 is fully assembled.

The optics assembly 130 is a generally planar support having at least a top face 148 and a bottom face 150. The bottom face 150 is configured to receive illumination from the LEDs 135, 137 and the optics assembly 130 directs the illumination to one or more sampling areas 152 on a first 154 and second 156 assay strip. The top face 148 of the optics assembly is also configured to transmit the diffusely reflected optical radiation returning from the sampling areas 152 to one or more of the zone detectors 138, 140.

The assay strips 154 and 156 mount in strip carriers 158 and 160 respectively. The carriers 158, 160 mount to the top face 148 of the optics assembly to rigidly hold the assay strips 154 and 156 in position.

Meter 100 includes batteries 168 that power the PCB 128 and a liquid crystal display (LCD) 162. A desiccant 164 and an absorptive material 169, for excess sample volume overflow, are also enclosed in the housing 102.

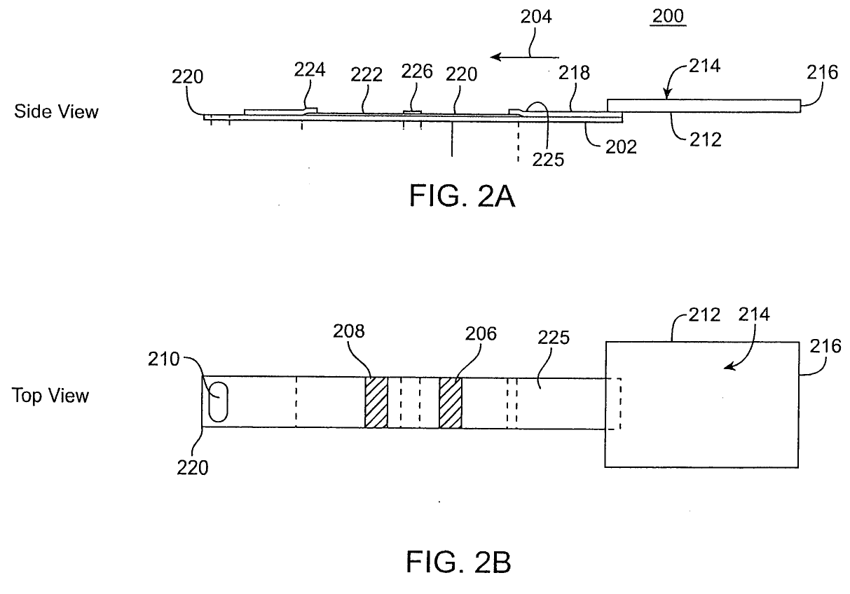

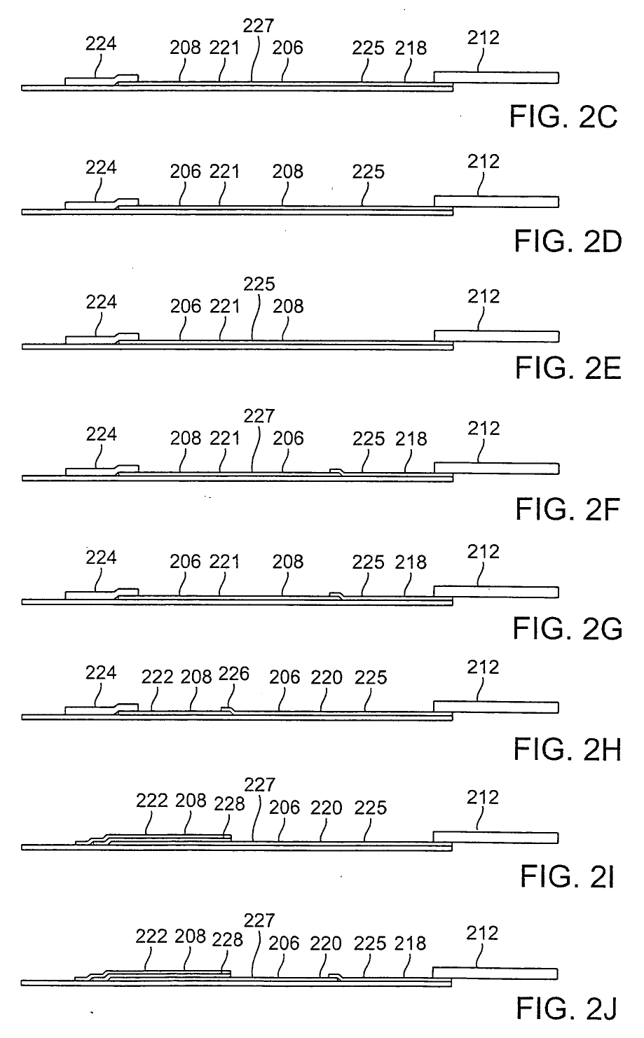

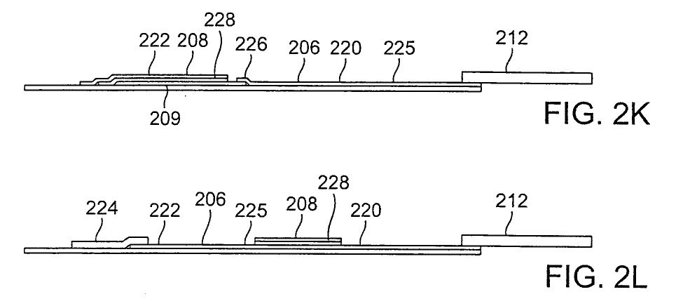

FIGS. 2A and 2B illustrate a laminated transport matrix 200 for a specific binding assay and a general chemical assay that is suitable for use in the preferred embodiment of the diagnostic device 100 described above (i.e. for use in assay test strips 154 and 156). In this embodiment of the invention, there are four distinct pieces of porous material in the fluid migration path of the transport matrix 200, each of which are laminated to a backing 202 made of a suitable plastic like PET in precise alignment with each other. FIG. 2A shows a longitudinal cross-section (side view) along the fluid migration path while FIG. 2B shows a corresponding top plan view. The sample wicks laterally in the direction as indicated by arrow 204 along the transport matrix 200 and into a first detection zone 206 and a second detection zone 208, respectively. The transport matrix 200 is held in alignment by a pin that fits into a sprocket hole 210 and by guides that fit against the sides of the strip.

The transport matrix 200 includes a sample pad 212 for receiving the sample through the inlet port (not shown) on the topside 214 of the pad 212 at the proximal end 216 of the transport matrix 200. In the example of using the diagnostic device illustrated in FIG. 1, the sample pad, preferably not physically attached to the rest of the assay strip, receives the sample and divides it between two separate transport matrixes 154, 156.

In an optional preferred embodiment, transport matrix 200 preferably includes a first detection zone pad 220 made of material such as nitrocellulose that has a uniform thickness of about 70 to about 240 μm, and preferably about 135 to about 165 μm. The wicking rate should be in the range of about 0.1 to about 0.6 mm/sec over about 4 cm, and preferably about 0.2 to about 0.4 mm/sec as a mean value. The opacity of the material is preferably such that any backing material is not visible or, alternatively, the backing material may be a white, reflective material such as white PET. In some cases, a black backing material may be preferred. The material should also have a reasonable dry and wet strength for ease of manufacturing. In the case of specific binding assays or other specific binding assays where a proteinaceous moiety must be non-diffusively immobilized on the membrane, the material should have a high capacity for protein adsorption in the range of about 1 to 200 μg/cm², and preferably 80 to 150 μg/cm².

In various preferred embodiments, transport matrix 200 preferably includes multiple segments of different materials that are in fluid communication with one another. The multiple segments of materials provide flexibility for the material of each segment to be optimized for a particular function. A multi-segmented transport matrix can advantageously avoid using a “compromise” material that can perform all the required test functions, although not with optimal results. (However, the transport matrix can instead be formed from a single continuous sheet of material that can perform all the required test functions). Fluid communication includes moving and/or traversing the sample in a lateral flow across the transport matrix by allowing the sample to flow through the plane and/or normal to the plane of the transport matrix. As further contemplated by the present invention, this two- or three-dimensional fluid communication movement through the plane and/or normal to the plane of the transport matrix can occur in sequence or simultaneously.

In one preferred embodiment, the sample pad 212 is preferably made of CytoSep No. 1660 or 1662 from Gelman Sciences that is cellulose and glass fiber composite material. The sample pad has approximately square dimensions of about 7 to 10 mm with a thickness of about 0.012 to 0.023 inch. Another material that is suitable is Ahlstrom filtration material grade 1281 which has a composition of about 90% cellulose fiber and 10% rayon with traces of polyamide wet strength resin and polyacrylamide dry strength resin. It has a basis weight of 70 g/m² and a thickness of about 0.355 mm.

The sample pad 212 attaches to and is in fluid communication with two transport matrices 154, 156 previously illustrated in FIG. 1. The sample flows from the sample pad 212 to a conjugate pad 218 that, in one preferred embodiment, is made of cellulose acetate for diffusively immobilizing a conjugate of anti-HbA1c with an indicator. The conjugate pad 218 may be about 7 mm long and 3 mm wide with a thickness of about 0.005 to 0.010 inch. The conjugate pad 218 may be attached by adhesive to a PET backing. Another suitable material for the conjugate pad 218 is Accuwik No. 14-20 from Pall Biosupport.

In one preferred embodiment, the diffusively immobilized conjugate 225 disposed on conjugate pad 218 may comprise anti-HbA1c with an indicator. Other possibilities for conjugate 225 include adsorption of anti-conjugate antibodies (i.e.: materials that bind to the conjugate regardless of whether the conjugate binds to anything else). Specific examples may include, but are not limited to, (1) impregnation with a material that binds to and immobilizes the conjugate, (2) an antibody directed against the conjugate, and (3) a polymer capable of bridging between and immobilizing conjugate microparticles.

The conjugate pad 218 overlaps and is in fluid communication with first detection zone pad 220. The first detection zone pad 220 is about 7 mm long and about 3 mm wide with a thickness of about 0.006 to about 0.008 inch. The first detection zone pad 220 allows the sample 112 to flow across the first detection zone 206 towards the distal end 220 of the transport matrix.

In preferred aspects of the invention, conjugate 225 is preferably located as close as possible to the overlap of conjugate pad 218 and detection (i.e. capture) zone pad 220. An advantage positioning conjugate 225 as close as possible to first detection zone pad 220 is that it prevents color streaking therein. Specifically, when the fluid sample first reaches conjugate 225, its viscosity increases. Thus, the fluid sample and conjugate mixture tends to initially gather at on conjugate pad 218 right next to its overlap with first detection zone pad 220. Then, the fluid sample and conjugate mixture spills over onto the first detection zone pad 220 in a manner that is uniform laterally across the width of the first detection zone pad 220.

The first detection zone pad 220 overlaps and is in fluid communication with a second detection zone pad 222. The second detection zone pad 222 is, in one embodiment, made from a nylon membrane such as Immobilon Nylon+, 0.45 um, from Millipore or Biodyne C from Pall Gellman, which has uniform opacity that is retained after impregnation with indicator and enzyme mixtures and subsequent drying. The second detection zone pad 222 is about 7 mm long and about 3 mm wide with a thickness of about 0.006 to about 0.008 inch. It allows the sample 112 to flow across the second detection zone 208 towards the distal end 220 of the transport matrix.

The junction 226 of the first detection zone pad 220 and the second detection zone pad 222 effectively traps the indicator bound conjugate. Thus, the indicator diffusively bound in the conjugate pad 218 is prevented from entering the second detection zone pad 222. Alternately, the sequence of the first and second detection zones may be reversed. In this case, the indicator conjugate 225 diffusively immobilized in the conjugate pad 218 washes through the first detection zone pad 220 (which may comprise a non-specific chemistry measurement zone for total hemoglobin), to the second detection zone pad 222 (which may comprise a specific binding assay zone that captures the indicator bound conjugate).

The second detection zone pad 222 overlaps and is in fluid communication with a sample absorbent pad 224 that allows the sample to flow across the second detection zone 206 towards the distal end 230 of the transport matrix.

A variety of different embodiments of the present transport matrix 200 are included within the scope of the present invention. FIGS. 2C to 2L show examples of various embodiments of the present transport matrix 200. Each of these exemplary embodiments have unique features and advantages, as will be described below. It is to be understood that the present transport matrix 200 is not limited to the specific embodiments shown in FIGS. 2A to 2L. Other transport matrix systems may be incorporated, all keeping within the scope of the present invention.

FIG. 2C is a side view of an alternative transport matrix employing a single membrane material with a specific binding assay zone positioned upstream of a general chemical assay zone. Specifically, a single detection zone pad 221 is shown. Detection zone pad may be made of nitrocellulose, but is not so limited. Conjugate 225 is disposed on detection zone pad 221 at the location as shown. In one preferred method of manufacture, conjugate 225 is applied by atomizer spray as a stripe onto the top of detection zone pad 221.

A fluid sample 112 (FIG. 1) is received onto sample pad 212. The fluid sample then wicks through transport matrix 220 (in direction 204) passing through conjugate 225. Thereafter, the sample passes first through the first detection zone 206 and then through the second detection zone 208. Any remaining conjugate is trapped at conjugate removal zone 227 before it has a chance to reach the second detection zone 208. Excess fluid sample is then simply washed into sample absorbent pad 224.

FIG. 2D is similar to FIG. 2C, but has the sequence of the specific binding assay zone 206 and the general chemical assay zone 208 reversed.

A primary advantage of the systems of FIGS. 2C and 2D is that they only require a single membrane on which both a specific binding assay and a general chemical assay are performed. The use of a single membrane eliminates the flow non-uniformities that can be introduced by small variations in membrane overlap dimensions. The lack of an overlap between the conjugate zone and detection zones also increases the efficiency with which the conjugate is washed through the strip.

FIG. 2E is similar to FIG. 2D, but conjugate 225 is instead initially disposed between general chemical assay zone 208 and specific binding assay zone 206. A particular advantage of this embodiment of transport matrix 200 is that no conjugate 225 passes through the general chemical assay zone 208. (In contrast, the embodiment in FIG. 2A used an overlap of membranes at junction 226 to prevent conjugate 225 from entering general chemical assay zone 208.) This configuration solves the problem of conjugate interfering with the reaction (or detection) performed in the general chemistry assay zone. Since no overlap at junction 226 is needed, nor is a chemical conjugate trap 227 potentially needed, the uniformity of liquid flow is preserved, and the risk of interference with the general chemistry from any chemical conjugate trap is avoided.

FIG. 2F shows an embodiment of transport matrix 200 in which conjugate 225 is disposed on a conjugate pad 218; and both the specific binding assay zone 206 and the general chemical assay zone 208 are disposed on a single detection zone pad 221.

FIG. 2G is similar to FIG. 2F, but has the sequence of the specific binding assay zone 206 and the general chemical assay zone 208 reversed.

A primary advantage of the systems of FIGS. 2F and 2G is that they only require a single membrane on which both a specific binding assay and a general chemical assay are performed. In addition, by employing a conjugate pad 218, conjugate 225 can be applied near the overlap with single detection zone pad 221 to prevent streaking therein, in the manner as was described above. Since many conjugate pad materials are of a relatively coarse nature, they are vulnerable to non-uniformity of liquid flow. Placement of the conjugate 225 near the overlap avoids this risk.

FIG. 2H shows an embodiment of transport matrix 200 in which conjugate 225 and specific binding assay zone 206 are both disposed on first detection zone pad 220; and general chemical assay zone 208 is disposed on second detection zone pad 222. Overlap 226 traps the conjugate 225, thus ensuring that conjugate 225 does not reach second detection zone pad 222 (and thus does not interfere with the general chemistry assay, nor with the reading of the general chemistry assay performed therein).

FIG. 2I is a side view of an alternative transport matrix 200 having a first detection zone pad 220 with a specific binding assay zone 206 thereon; and a second detection zone pad 222 with a general chemical assay zone 208 thereon. A spreader/treatment/filtration layer 228 is disposed under second detection zone pad 222. Spreader layer 228 operates to assure lateral distribution of the sample prior to migration into the detection zone pad 222. A conjugate removal zone 227 is formed by application of a material that binds to or causes aggregation of the conjugate and operates to immobilize it, thus preventing migration into the second detection zone pad 222. This embodiment of transport matrix 200 is ideally suited for detection of creatinine, but is not so limited. Materials that are suitable for a conjugate removal zone include but are not limited to chemically-modified membrane matrices, such as nylon modified to have positively or negatively charged functional groups, positively or negatively charged polymers such as polyethyleneimine or polyacrylic acid, and anti-conjugate antibodies.

FIG. 2J is similar to FIG. 21, but with conjugate 225 instead being disposed on a conjugate pad 218. As mentioned above, conjugate pad 218 can be used to prevent sample streaking.

FIG. 2K is similar to FIG. 2I, but with an additional layer 209 disposed under spreader layer 228. The junction 226 between first detection zone pad 220 and layer 209 acts as a conjugate trap, preventing the conjugate from reaching spreader layer 228 (and second detection pad 222).

FIG. 2L is a side view of an alternative transport matrix 200 having a spreader layer 228 disposed under first detection zone pad 220. General chemical assay zone 208 is disposed on first detection zone pad 220. Specific binding assay zone 206 is disposed on second detection zone pad 222.

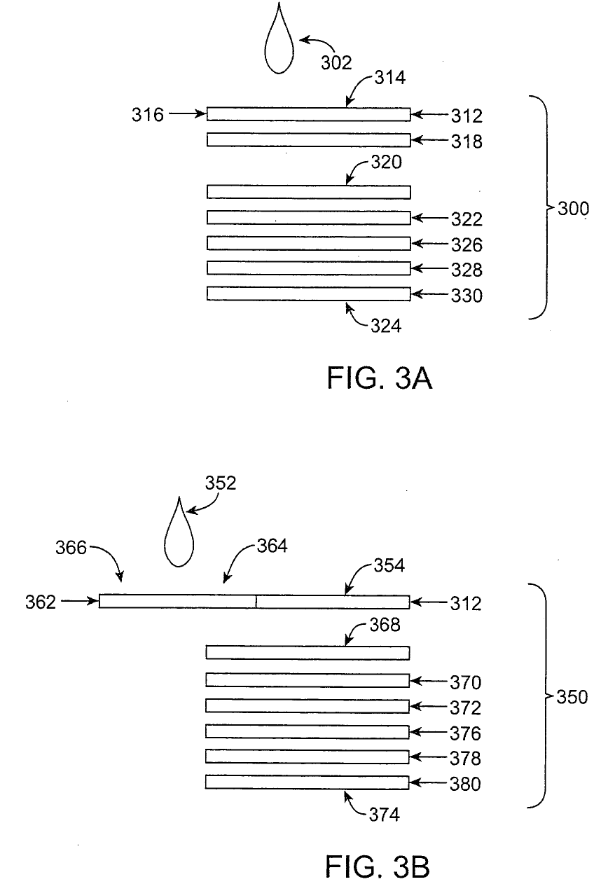

FIGS. 3A and 3B illustrate stacked transport matrices for a specific binding assay and a general chemical assay that are suitable for use in alternative embodiments of the preferred diagnostic device 100 described above. FIG. 3A shows an exploded side view of an alternate embodiment 300 of the transport matrix with the fluid communication path primarily in a transverse flow normal to the plane of the porous materials. In preferred embodiments, there are a plurality of distinct pieces of porous material in the fluid migration path of the stacked transport matrix 300, each of which are in fluid communication with each other either directly or through other porous materials, channels or fluid communication devices. The transport matrix 300 includes a sample pad 312 for receiving the sample 302 through the inlet port (not shown) on the topside 314 of the pad 312 at the proximal end 316 of the transport matrix 300. The sample pad 312 is preferably made of a cellulose and glass fiber composite material.

The sample pad 312 overlays and is in fluid communication with a conjugate pad 318 for a first analyte that may optionally be made of cellulose acetate for diffusively immobilizing a conjugate of anti-HbA1c with an indicator. The conjugate pad 318 overlays and is in fluid communication with a capture and first detection zone pad 320 for the first analyte that may optionally be made of a nitrocellulose substrate. The first detection zone pad provides a first detection zone (not specifically delineated in FIG. 3A) for the first analyte. With the preferred system of detection by optical reflection, the detection of the first analyte in the first detection zone pad can be significantly improved by optically isolating the first detection zone so that the loss of optical reflectance is minimized. Accordingly, the transport matrix 300 can optionally provide an optical isolation membrane 322 that will minimize the loss of reflected light through the porous materials at the distal end 324 of the transport matrix. The optional optical isolation membrane 322 is in fluid communication with the first detection zone pad 320 and allows the sample 302 to flow to a conjugate removal zone pad 326 that effectively traps the indicator bound conjugate and prevents it from entering any detection zones on the transport matrix distal to the first detection zone.

Optionally, a second optical isolation membrane 328 overlays and is in fluid communication with the sediment filtration zone pad 326. The sample 302 flows through the second optical isolation membrane 328 to the non-specific measurement zone pad 330 that is in fluid communication with the proximal pads and membranes. The measurement zone pad 330 may optionally be made of a plain nylon and has a uniform opacity that is retained after impregnation with indicator and enzyme mixtures and subsequent drying. The measurement zone pad 330 allows the sample 302 to flow across a second detection zone (not specifically delineated in FIG. 3A) towards the distal end 324 of the transport matrix. Separate measurements of the reflectance of detection zone pads 320 and 330 may be obtained by optically interrogating the top and bottom of the membrane stack, respectively.

FIG. 3B shows an exploded side view of another alternate embodiment 350 of the inventive transport matrix with the fluid communication path in both a lateral and a transverse flow parallel to and normal to the plane of the porous materials, respectively. Generally, there are a plurality of distinct pieces of porous material in the fluid migration path of the transport matrix 350, each of which are in fluid communication with each other either directly or through other porous materials, channels or fluid communication devices. The transport matrix 350 includes a sample pad 362 for receiving the sample 352 through the inlet port (not shown) on the topside 364 of the pad 362 at the proximal end 366 of the transport matrix 350. The sample pad 362 may optionally be made of a cellulose and glass fiber composite material.

The sample pad 362 abuts and is in fluid communication with a sample distribution pad 354 which divides the sample 352 between one or more additional transport matrices (not shown). The sample distribution pad 354 overlays a conjugate pad 368 for a first analyte that is preferably made of nitrocellulose for diffusively immobilizing a conjugate of anti-HbA1c with an indicator. The conjugate pad 368 overlays and is in fluid communication with a capture and first detection zone pad 370 for the first analyte preferably made of a nitrocellulose substrate. The first detection zone pad provides a first detection zone (not specifically delineated in FIG. 3B) for the first analyte.

The transport matrix 350 can optionally provide an optical isolation membrane 372 that will minimize the loss of reflected light through the porous materials at the distal end 374 of the transport matrix. The optional optical isolation membrane 372 is in fluid communication with the first detection zone pad 370 and allows the sample 352 to flow to a conjugate removal zone pad 376 that effectively traps the indicator bound conjugate and prevents it from entering any detection zones on the transport matrix distal to the first detection zone.

Optionally, a second optical isolation membrane 378 overlays and is in fluid communication with the sediment filtration zone pad 376. The sample 352 flows through the second optical isolation membrane 378 to the non-specific measurement zone pad 380 that is in fluid communication with the proximal pads and membranes. The measurement zone pad 380 is preferably made of a plain nylon and has a uniform opacity that is retained after impregnation with indicator and enzyme mixtures and subsequent drying. The measurement zone pad 380 allows the sample 352 to flow across a second detection zone (not specifically delineated in FIG. 3B) towards the distal end 374 of the transport matrix.

It is important to note that the present invention contemplates the use of any combination of lateral and transverse sample flow arrangements. The transport matrix may use alternating or successive pads, membranes or the like in a flow that is either parallel to or normal to the plane of those pads, membranes or the like.

One of the preferred embodiments of the present invention is to perform a quantitative test for HbA1c. In order to run a chemical test and a specific binding assay on the same lateral flow strip, one of the detection zones should read only one analyte. The measurement in the other detection zone may reflect a combination of the results from the two analytes. However, a method must determine the contribution of each analyte to the combined detection zone. For example, if Analyte 2 is an enzyme or a colored analyte, and Analyte 1 is a protein whose presence must be determined via an immunochemical reaction, detection zone 2 (e.g.: the general chemical assay zone) reads only Analyte 2, but detection zone 1 (e.g.: the specific binding assay zone) reads both Analytes 1 and 2. The concentration of Analyte 1 can be calculated by making a correction in the detection zone 1 measurement to account for the contribution of Analyte 2.

Detection zone 2 can be constructed in a variety of ways to block out any contribution of the detection zone 1 reaction. In a preferred embodiment, a striped protein capture zone and blue latex microparticles are used to perform the immunoreaction in detection zone 1 (i.e.: specific binding assay zone 206). Movement of the blue latex microparticles up the strip must be blocked, so that they would not be visible in detection zone 2 (i.e.: general chemical assay zone 208). In embodiments of the invention shown in FIGS. 2A, 2B, 2H, and 2K, a small pore size nylon membrane 222 or 209 with a positive charge was chosen as the capture zone of for blue latex microparticles. The highest positive charge coating yielded the best results with regard to a lack of chromatography of the sample as it flowed up the strip.

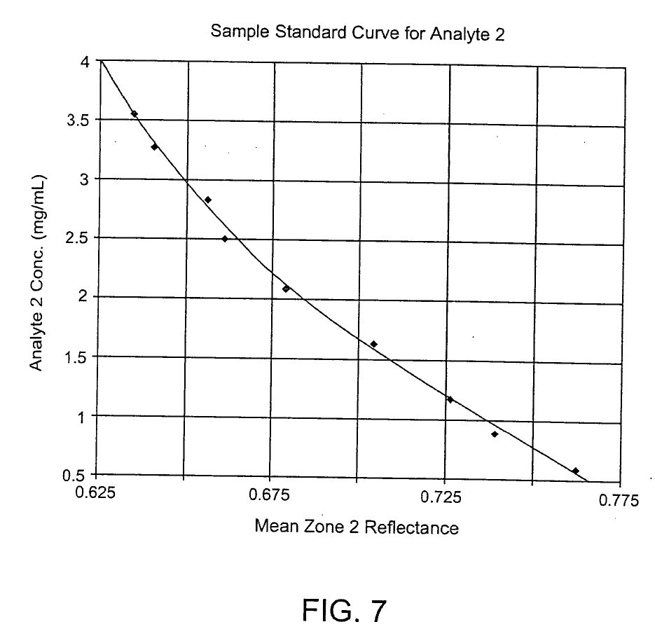

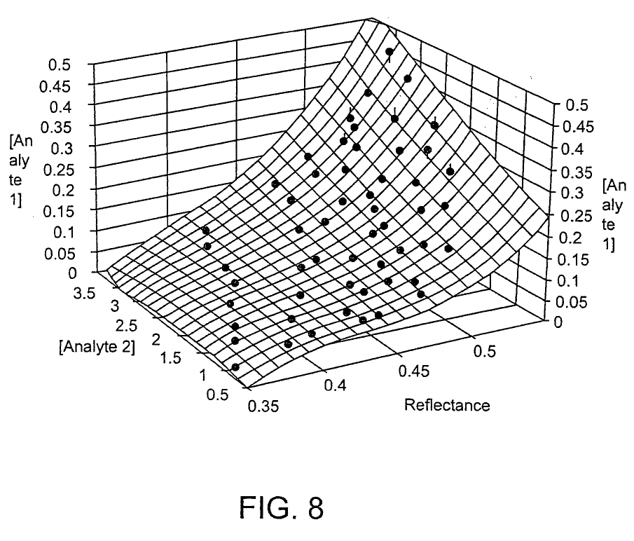

The concentration of Analyte 2 is determined from the reflectance in detection zone 2 as shown in FIG. 7. To correct for the contribution of Analyte 2 in detection zone 1, a mathematical algorithm was used to define the concentration of Analyte 1 as a function of the reflectance in detection zone 1 and the concentration of Analyte 2. This algorithm is graphed in FIG. 8. This algorithm was derived by assaying a series of Analyte 1 concentrations at a series of Analyte 2 concentrations, and determining the resulting detection zone 1 reflectance.

A diagnostic kit is included in the present invention for determining the levels of a first and a second analyte in a sample. The kit includes a sample receptacle containing a chemical indicator for performing a general chemical assay on the sample, by reacting with the second analyte to produce a detectable result, and a device as recited above. The term receptacle includes, and is not limited to, screw cap vials, snap cap vials, containers, pouches, and the like.

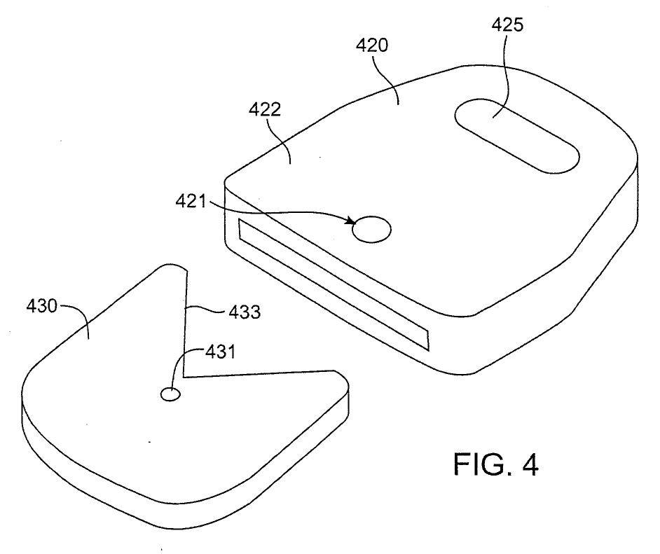

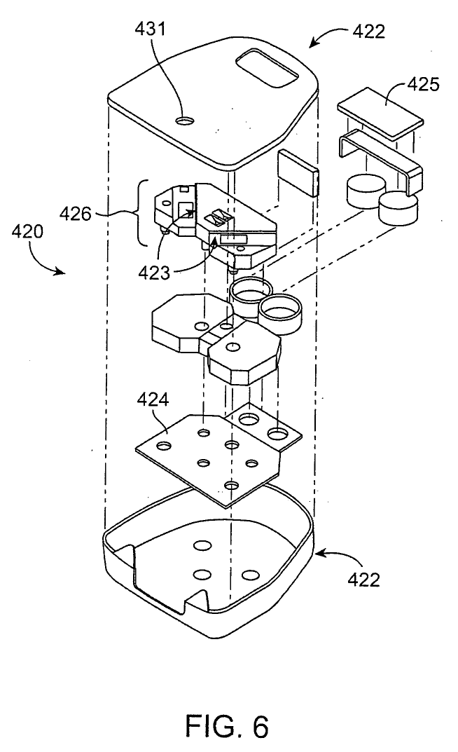

FIGS. 4 to 6 illustrate a preferred embodiment of the invention comprising a disposable cartridge 430 that is received into a multi-use meter 420. Meter 420 includes a housing 422 with a logic circuit 424 and an optical system 426 therein. A visual display 425 is disposed on the outside surface of housing 422. Cartridge 430 includes a sample pad 432; and at least one test strip 434 in contact with sample pad 432. As will be explained, cartridge 430 is receivable into the body fluid analyte meter 420 such that test strips 434 are each positioned to be read by the optical system 426 in housing 422.

Test strips 434 preferably comprise any of the embodiments of transport matrices 200, 300, or 350 as described above. Thus, assay test strips 434 function in the same manner as assay test strips 154 and 156 as described above. In a preferred embodiment, test strips 434 comprise a reagent which reacts with a blood sample to yield a physically detectable change which correlates with the amount of selected analyte in the blood sample. Most preferably, the reagent on each test strip reacts with the blood sample so as to indicate the concentration of hemoglobin A1c (HbA1c). Examples of detection systems appropriate for use in measuring hemoglobin A1c (HbA1c) are seen in U.S. Pat. Nos. 5,837,546; 5,945,345 and 5,580,794, incorporated by reference herein in their entirety for all purposes. It is to be understood, however, that the present invention is not limited to using such reagents and reactions. Other analytic possibilities are also contemplated, all keeping within the scope of the present invention.

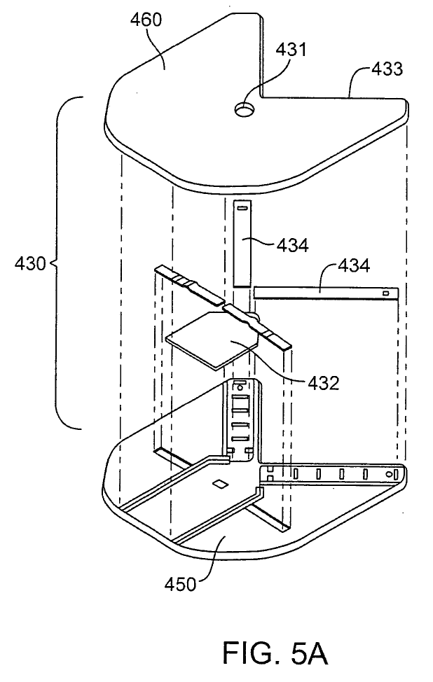

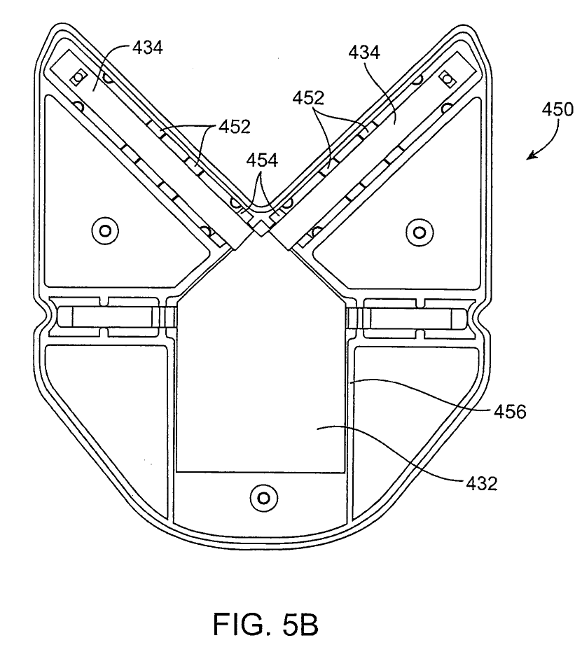

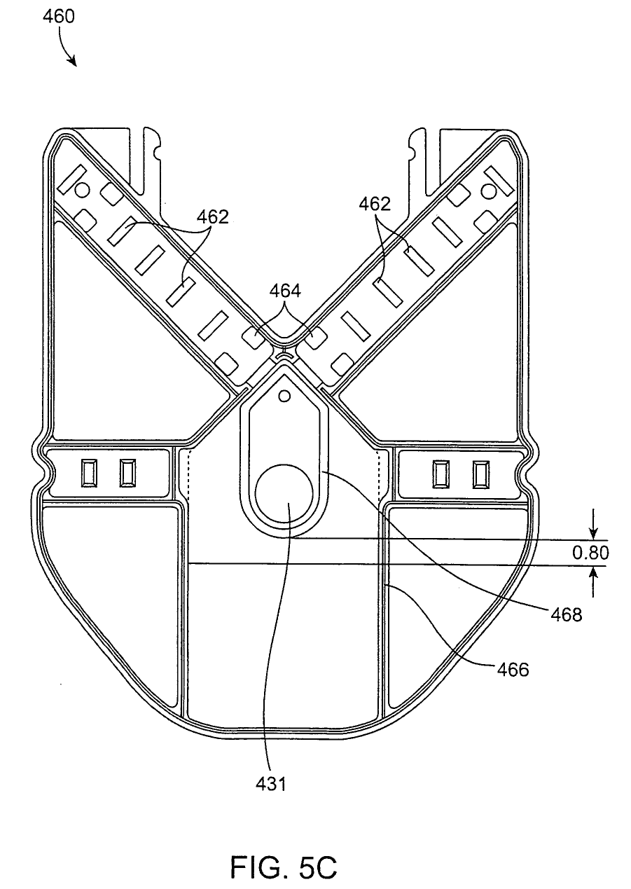

As can be seen in FIG. 5A, a pair of test strips 434 may be provided. In operation, a blood sample is first received through top hole 431 (in cartridge 430) and then drops directly onto sample pad 432. Each test strip 434 is in contact with sample pad 432 such that the blood sample wicks from sample pad 432 onto each of test strips 434. Thus, parallel reactions occur in the pair of test strips 434 between the blood and the reagent pre-embedded within or coating the test strips.

In alternate embodiments, hole 431 remains fully outside of meter 420 when cartridge 430 is received into meter housing 422. An advantage of this embodiment is that the blood sample never passes through meter 420, thus resulting in a system with decreased potential for contamination.

Together, the bottom 450 and top 460 of cartridge 430 sandwich sample pad 432 and sample strips 434 holding test strips 434 firmly in position. Various features shown in the interior surface of the cartridge bottom 450 and cartridge top 460 serve to retain test strips 434 in position so that they will line up properly with the light source and detection lenses in the optics module (system 426), as follows.

As can be seen in FIG. 5B, sample pad 432 and test strips 434 are positioned in bottom 450. Fluid on sample pad 432 wicks onto test strips 434 in parallel. A series of support ribs 452 extend upwardly from bottom 450 and are positioned below test strips 434. As can be seen in FIG. 5C, a series of support ribs 462 extend downwardly from top 460 and are positioned above test strips 434. Support ribs 452 and 462 function to gently squeeze test strips 434. This is advantageous in ensuring complete fluid transfer from one portion of the test strip to the next. Specifically, such support ribs can be used to gently squeeze the overlap of conjugate pad 218 and first detection zone pad 220, the overlap of first detection zone pad 220 and second detection zone pad 222 (at junction 226) and sample absorbent pad 224. (See FIG. 2A). In preferred embodiments, ribs 452 and 462 extend laterally across test strips 434, thereby restraining any left side/right side flow biases in test strips 434. In addition, support ribs 454 and 464 can be used to squeeze together the contact between sample pad 432 and test strips 434, thus ensuring easy fluid transport therethrough.

Additional fluid control features in cartridge 430 may include pinch walls 456 and 466 around sample pad 432 to prevent fluid sample from splashing around the interior or cartridge 430. A further pinch wall 468 around aperture 431 can be used to keep the fluid sample at a preferred location (adjacent to the ends of test strips 434).

As shown in FIG. 5D, an optical system 426 includes optical reader(s) which measure/detect the reaction occurring on each of test strips 434. For example, optical system 426 can be used to detect the blood/analyte reaction occurring on strip 434 which correlates to hemoglobin A1c (HbA1c) concentration in the blood sample. Logic circuit 424 analyzes the results of the optical detection and then visually displays the result on visual display 425 on housing 422. After this concentration result has been displayed, cartridge 430 is then removed from meter 420, and discarded. When a new test is to be performed, a new cartridge 430 is received into housing 422 in meter 420.

As can also be seen, when cartridge 430 is received fully into meter 420, test strips 434 in cartridge 430 are positioned to be read by an optical system 426. In addition, when cartridge 430 is received into meter 420, sample receiving aperture 421 (in cartridge 430) is positioned directly under sample receiving aperture 421 (in meter 410). Thus, when a blood sample is dropped through hole 421, it passes through hole 431, and lands on sample pad 432. From there, the blood sample wicks onto test strips 434, and the reaction in the test strips commences. The results of this reaction are measured by optical system 426 which conveys information to logic circuit 424 which in turn displays the result (e.g. the hemoglobin A1C concentration) on visual display 425 for a user to see. This is advantageous in that any blood fluid sample entering meter 410 (through sample receiving aperture 421) is contained in disposable cartridge 430. Thus, blood/fluid samples never contaminate the interior workings of meter 420.

As can also be seen, when cartridge 430 is fully received into housing 422, the V-shaped notch 433 in cartridge 430 is received against a V-shaped stop 423 adjacent to optical system 426 within housing 422. As such, when cartridge 430 is fully received into housing 422, each of test strips 434 are positioned directly above (or alternately, below) optical reader 426. It is to be understood that the V-shaped stop 423 may simply comprise an edge of optical system 426 as shown, or it may instead comprise an additional element (e.g.: wall or inner surface) of the invention.

As can be seen, V-shaped stop 423 and V-shaped notch 433 operate together to center and aligning cartridge 430 within housing 420. It is to be understood that alternate geometries may be employed, all keeping within the scope of the present invention. For example, a V-shaped notch may instead be located on housing 422 and a complementary fitting V-shaped edge or wall may instead be positioned on cartridge 430. Many alternate geometries are possible, all keeping within the scope of the present invention.

The “V” shape of cartridge 430 lines up exactly with the raised “V” edges on the optics module (i.e.: adjacent to, or on, optical system 426) to assure proper alignment. Optionally, detents may be provided in the side edges of cartridge 430 that will match spring-like features in meter 420 to provide for a positive snap-in action when cartridge 430 is properly placed into meter 420.

Optical system 426 operates by detecting a measurable change in test strip 434 when test strip 434 is exposed to a blood sample. In the optional embodiment shown, a pair of test strips 434 are used and read by a separate optical reader in system 426. The advantage of this embodiment of the invention is that a more accurate and precise result is obtained by simultaneously performing the same reaction on both test strips 434, and then comparing the result. It is to be understood, however, that the present invention is not limited to embodiments of the invention with two test strips 434. Rather, one, two or more test strips are contemplated, all keeping within the scope of the present invention. Moreover, a plurality of test strips, with different test strips comprising different analytes for testing different assays is also contemplated to be within the scope of the present invention.

In accordance with the present invention, analyte calibration information may be pre-stored in logic circuit 424. For example, since all of the disposable cartridges 430 packaged with any given multi-use meter 420 will be from the same manufacturing lot, their calibration parameters may be pre-programmed into meter 420's memory. A used cartridge 430 is simply removed from meter 420 after the test is completed. Meter 420 can then be re-used with a fresh cartridge 430 from the same batch. Each cartridge 430 may optionally be individually foil-wrapped to assure stability (protection from moisture). Alternatively, analyte calibration information may be pre-stored in cartridge 430 (and then be read by logic circuit 424 when cartridge 430 is received into meter 420). This alternate embodiment would permit a single meter 420 to be used with cartridges 430 made from various batches of cartridges. Such an embodiment would considerably extend the useful life of meter 420.

In an optional preferred embodiment of the invention, an identification tag 480 is mounted on the exterior of cartridge 430. Such identification tag may comprise an optical machine readable code that is read by an appropriately positioned detector during cartridge insertion. For example, a barcode. Alternately, identification tag 480 may be an RF tag that is disposed within cartridge 430.

Optionally, an autostart circuit configured to activate the meter when the sample is applied to the cartridge, or the cartridge is received into the housing, may also be provided. An example of such an autostart system is seen in one or more of U.S. Pat. Nos. 5,837,546; 5,945,345 and 5,580,794, incorporated by reference herein in their entirety for all purposes.

As mentioned briefly above, an integrated sampler device may optionally be used to initially introduce the blood sample through hole 421. Such integrated sampler may be used to first mix the blood sample with a sample dilution buffer prior to introducing the blood through hole 421 and into cartridge 430. In one embodiment of the integrated sampler, the sample dilution buffer may be contained in a reservoir in the integrated sampler. The integrated sampler may optionally be received into a port (hole 421) in meter 420.

### EXAMPLE 1

A series of studies was performed to evaluate the preferred device for measuring HbA1c in terms of conventional laboratory (nonclinical) performance characteristics, including assay linearity (recovery) and hematocrit tolerance, as well as selected user manipulations that may be encountered in the physician's office laboratory (POL) or home settings. The FDA's Guidance Document Review Criteria for Assessment of Glycohemoglobin (Glycated or Glycosylated) Hemoglobin In Vitro Diagnostic Devices, Center for Devices and Radiological Health (HFK-440 NChace/chron Feb. 24, 1991 Version Sep. 27, 1991) was taken into account when these studies were designed.

Nonclinical performance studies were conducted in either of two ways. The first method utilized a fully assembled preferred embodiment of the above described assay device 100 HbA1c units containing previously “uploaded” calibration coefficients. In this method, samples were applied to the units for evaluation and the data subsequently downloaded to a personal computer. To accomplish downloading, the units were placed into “docking stations” that mechanically and electrically connected them to a standard computer via the preferred device's communication port and a serial port adapter. The downloaded reflectance values were, in turn, transferred to and displayed in an EXCEL® spreadsheet (Microsoft Inc., Redmond, Wash.) and converted to units of % HbA1c. In this scenario, downloading could take place at any time after the reactions were complete. “Downloadable” information is retained in device units for as long as the batteries are functional. Following the downloading step, the units were discarded.

The second method utilized “reusable” units. In this method, HbA1c test strips were placed into units and clamped shut on the docking station as described above. Samples were applied to the units for evaluation, and the reflectance data automatically downloaded in a fashion similar to that for the method described above, except that it took place in “real” time.

The linearity (recovery) study followed a modified NCCLS protocol (NCCLS Document EP-6-P Vol. 6, No. 18, “Evaluation of Linearity of Quantitative Analytical Methods”). Clinical samples representing low and high % HbA1c were identified. “Low” was defined as samples with analyte concentrations at or near the low end of the device's HbA1c's dynamic range, and “High” was defined conversely. The low and high samples were mixed and labeled into nine preparations as shown in Table 1 in order to assess linearity for % HbA1c.

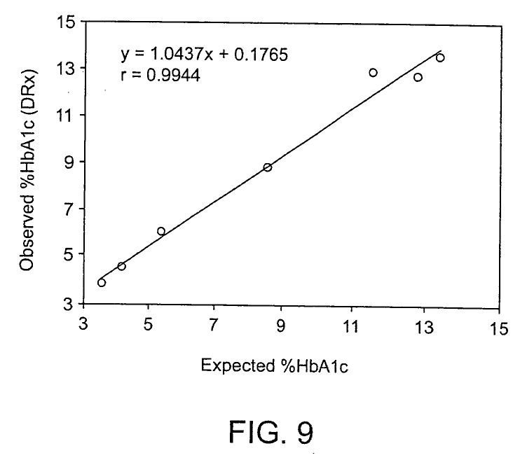

Samples were tested in replicates of five for all testing, except for the neat samples (Mixtures 1 and 9) that were tested in replicates of 10. The observed % HbA1c means were compared to the expected results and analyzed in terms of percent recovery. Linear regression (FIG. 9) was performed to assess linearity and to obtain a correlation coefficient. The results from the testing of the pure samples (Mixtures 1 and 9) were used as the reference values from which the expected values were calculated. Percent recovery was calculated as 100 times the observed value divided by the expected value. Summary recovery results are presented in Table 1.

The data demonstrate that the % HbA1c assay is linear between 2.5 and 14.5 % HbA1c as shown graphically in FIG. 9. The dynamic range for % HbA1c is thus 3% to 15% (rounding to the nearest whole number).

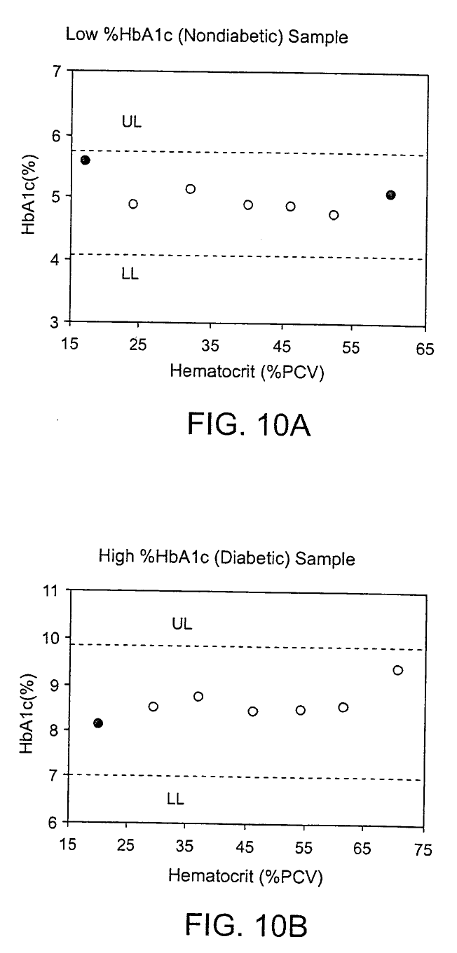

Another study was conducted to determine the impact of different hematocrit levels on the performance of preferred assay device for HbA1c. The results of this study are shown in tabular form in Table 2 and graphically in FIGS. 10A and 10B. Whole blood samples at two % HbA1c levels (diabetic and nondiabetic) were adjusted to differing levels of hematocrit by centrifugation and resuspension of red cells in autologous plasma. These were then tested by standard procedures. Five replicate analyses were performed for each test condition and for each control (native) sample. Upper and lower limits (UL and LL) were calculated for the 99% confidence interval for total error (±[|bias|+3×SEM]) from the native sample value. PCV refers to packed cell volume and SEM refers to the standard error of the mean. In FIGS. 10A and 10B, upper and lower limits (UL and LL) are shown as dashed lines (----). The data points that are solid black (•) are from samples not within the specified total hemoglobin range for the inventive HbA1c test device.

The results in parentheses in Table 2 represent samples where the total hemoglobin fell outside the specified total hemoglobin limits for the assay (68-200 mg/mL). Consequently, they would not be reported on the device's LCD and the user would obtain an out-of-range (OR) error code. They are reported here for information only.

These results indicate that all samples within the specified total hemoglobin tolerance for the inventive assay device for HbA1c (68-200 mg/mL) yielded equivalent values. All values fell within the 99% confidence interval for total error from the mean control (native sample) value. The hematocrit range for the assay device for HbA1c is thus 20% to 60% PCV. As shown above, samples in this range will give reliable results.

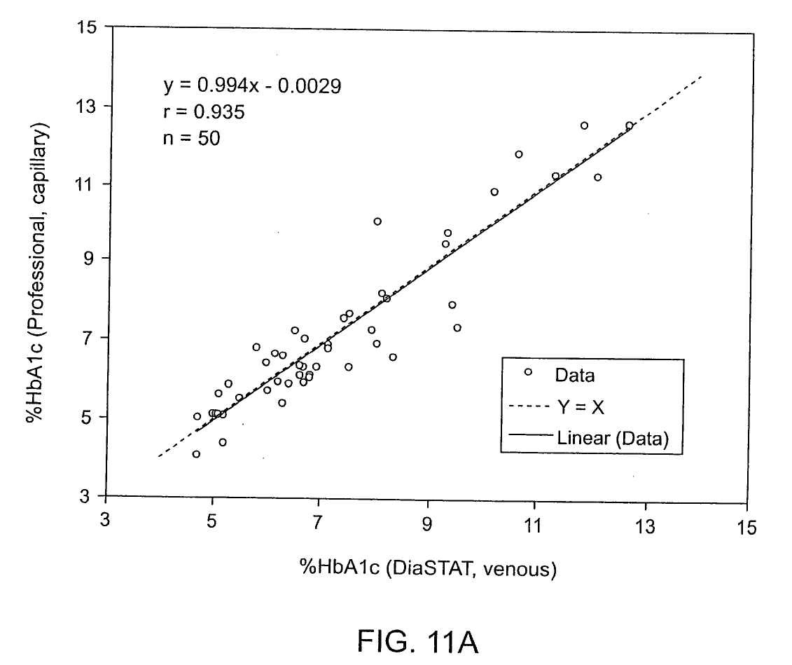

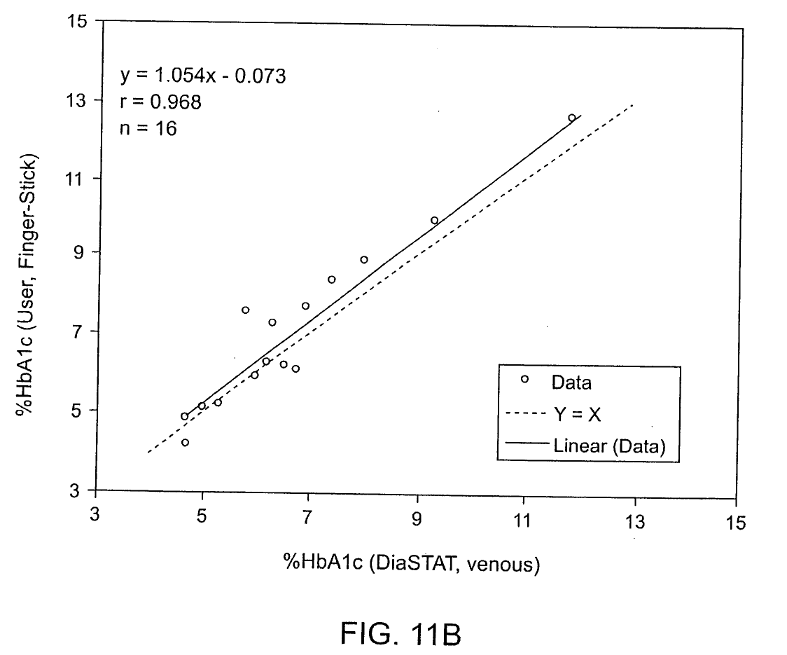

FIG. 11A shows the test data from the inventive assay device run by professionally trained medical personnel using finger-stick patient samples. The percentage HbA1c results obtained in these studies were substantially equivalent to the results obtained with the certified laboratory test method known as DiaSTAT. FIG. 11B shows a graph of the data from self testing patients using the assay kit of the present invention. Again, the results obtained by non-medical personnel were comparable to the certified laboratory test method DiaSTAT.

The imprecision in the clinical decision range over two days of testing was initially as low as 5.0% CV as seen in the data presented in Table 3 below. Performance did not degrade substantially when testing was expanded day-to-day over 5 days as shown in Table 4 below.

### EXAMPLE 2

Preparation of the general chemical portion of a strip for the detection of creatinine (e.g.: as shown in FIGS. 2I, 2J, 2K and 2L can be made in accordance with the present invention using three separate processes. The following exemplary processes were used in the preparation of the general chemical zone.

The first process is to impregnate a roll of nylon membrane with a suspension of 15% titanium dioxide. This suspension is prepared by mixing in a high-speed mixer the following components in successive order: 0.25 g/mL 1% PVA 186K; 0.5966 g/mL distilled water; 0.00075 g/mL tripolyphospate; 0.00075 g/mL fumed silicon dioxide; and 0.15 g/mL titanium dioxide. After coating, the membrane is dried at 37° C. for 10 minutes and allowed to equilibrate under dry room conditions for at least 8 hours prior to the second coating.

The second process is to stripe an enzyme solution using a platform striper with a metering pump such as those made by IVEK of North Springfield, Vt. Other applicators suitable for use with the present invention include, but are not limited to, fountain pens, pad printers, pipettes, air brushes, metered dispensing pumps and tip systems, or the like. Other applicators which accurately measure the reagents onto appropriate zones of the predetermined distribution are also suitable. The enzyme solution is striped 5.25 mm from one edge of the processed nylon material impregnated with titanium dioxide. The solution includes: 1000 U/mL creatinine amidinohydrolase; 4000 U/mL creatine amidohydrolase; 1000 U/mL sarcosine oxidase; 1000 U/mL horse radish peroxidase; 22.92 g/L TES; 10 g/L sucrose; 10 g/L Triton X-100; and 0.1 g/mL xanthan gum.

The final process is to stripe an indicator solution over the enzyme-striped zone. This coating process is analogous to the one described above. The indicator solution includes: 0.0620 g/mL bis-MAPS-C3; 0.25 mL/mL isopropyl alcohol; 0.005 g/mL sucrose; 0.05 mL/mL Surfactant 10G; 0.05 mL/mL 20% PVP 40K; and 0.65 mL/mL Milli-Q water.

The metering membrane layer is prepared by impregnating a roll of nylon membrane about 7 mm wide in a buffer solution consisting of 250 mM MOPSO pH 7.5; and 0.5% (W/V) PVA 186K. This impregnating process is similar to the dip and dry process for the titanium dioxide.

The creatinine zone 208 of FIGS. 2I to 2L is prepared according with the following amendments. The nylon shown in FIGS. 2I to 2L comprises a metering membrane layer (approximately 5×3 mm). The enzyme membrane (2.18×3 mm) is attached to a white PET backing with adhesive (ARcare 8072, 22.46×3 mil) in the order of sequence illustrated in FIGS. 21 to 2K.

Conditions yielding the best proportionality between 15 and 30 mM creatinine standards (in K/S) were selected as optimal. The assay was run by loading 60 μL of a known creatinine standard into a diagnostic device similar to that described in FIG. 1. The progress of the enzymatic reaction was monitored until an endpoint was obtained which was typically 3 to 5 minutes after application of the sample. Final R/R₀ values for the test zone were obtained by picking the minimum value over the period examined.

For determination of creatinine, two duplicate strips can be placed in a breadboard reflectance reader that can analyze disposable strips. The reader takes end point reflectance readings for both test zone 1 and test zone 2. A calibration curve generated for creatinine (test zone 2) serves to determine the unknown concentration of the analyte. A calibration curve similar to that produced for determining total hemoglobin (“Analyte 2” in FIG. 8, above) can be made for test zone 2.

Test zone 1 can be constructed to perform a specific binding assay for albumin for the detection and measurement of microalbuminuria or for another analyte of interest.

Numerous modifications and variations of the present invention are possible in light of the above teachings. It is therefore to be understood that within the scope of the appended claims, the invention may be practiced otherwise than as specifically described herein.

TABLE 1
Percent HbA1c recovery.

| Mixture | Sample Proportion | | Observed | | Expected | Recovery |
|---|---|---|---|---|---|---|
| No. | Low | High | % HbA1c | N | % HbA1c | (%) |

| | | | | | | |
|---|---|---|---|---|---|---|
| 1 | 10 | — | 2.46 | 10 | — | — |
| 2 | 9 | 1 | 3.75 | 5 | 3.62 | 103.7 |
| 3 | 8.5 | 1.5 | 4.45 | 5 | 4.20 | 105.9 |
| 4 | 7.5 | 2.5 | 6.00 | 5 | 5.37 | 111.7 |
| 5 | 5 | 5 | 8.86 | 5 | 8.34 | 106.2 |
| 6 | 2.5 | 7.5 | 12.95 | 5 | 11.38 | 113.8 |
| 7 | 1.5 | 8.5 | 12.85 | 5 | 12.61 | 101.9 |
| 8 | 1 | 9 | 13.70 | 5 | 13.23 | 103.5 |
| 9 | — | 10 | 14.48 | 10 | — | — |
| Mean | | | | | | 106.7 |

TABLE 2
SUMMARY HEMATOCRIT TOLERANCE RESULTS.

| | | DRx ® | DRx ® | Lower | |
|---|---|---|---|---|---|
| | Hematocrit | (Total | (% | Limit (% | Upper Limit |
| Sample | (PCV) | Hb) | HbA1c) | HbA1c) | (% HbA1c) |
| Low | (60) | (204.8) | (5.1) | 4.1 | 5.7 |
| % HbA1c | | | | | |
| (nondiabetic) | | | | | |
| | 52 | 184.6 | 4.7 | | |
| | 46 | 162.4 | 4.9 | | |
| | 40 | 141 | 4.9 | | |
| | 32 | 122.3 | 5.1 | | |
| | 24 | 86.5 | 4.9 | | |
| | (17) | (64.8) | (5.6) | | |
| High | 70 | 193.8 | 9.4 | 7.0 | 9.8 |
| % HbA1c | | | | | |
| (diabetic) | | | | | |
| | 61 | 189.2 | 8.6 | | |
| | 54 | 169.7 | 8.5 | | |
| | 46 | 127.7 | 8.4 | | |
| | 37 | 113.1 | 8.7 | | |
| | 29 | 93.4 | 8.5 | | |
| | (20) | (58.8) | (8.1) | | |

TABLE 3

| | | | | |
|---|---|---|---|---|
| | | % HbA1c | | |
| Level | Mean | Std Dev | CV (%) | N(2 days) |

| | | | | |
|---|---|---|---|---|
| 1 | 5.9 | 0.29 | 4.97 | 15 |
| 2 | 10.3 | 0.80 | 7.81 | 15 |

TABLE 4

% HbA1c

| Level | Mean | Std Dev | CV (%) | N(5 days) |
|---|---|---|---|---|
| 1 | 6.12 | 0.47 | 7.66 | 30 |
| 2 | 11.34 | 1.02 | 8.95 | 30 |

1. A combination body fluid analyte meter and cartridge system, comprising:
(a) a body fluid analyte meter, comprising:
a housing;
a logic circuit disposed within the housing;
a visual display disposed on the housing; and
a measurement system disposed within the housing; and
(b) a cartridge, comprising:
at least one lateral flow assay test strip, the lateral flow assay test strip comprising:
(i) a lateral flow transport matrix;
(ii) a specific binding assay zone on the transport matrix for receiving a fluid sample and performing a specific binding assay to produce a detectable response, and
(iii) a general chemical assay zone on the transport matrix for receiving the fluid sample and performing a general chemical assay to produce a detectable response;
wherein the cartridge is dimensioned to be receivable into the body fluid analyte meter such that the measurement system is positioned to detect the responses in the specific binding assay zone and the general chemical assay zone in the lateral flow assay test strip.

2. The system of claim 1, wherein the measurement system is an optical measurement system.

3. The system of claim 2, wherein the optical measurement system measures reflectance.

4. The system of claim 1, wherein the cartridge is configured to be received into the meter prior to the introduction of the fluid sample into the cartridge.

5. The system of claim 1, wherein the cartridge is a single-use disposable device.

6. The system of claim 1, wherein the body fluid analyte meter is a multi-use device.

7. The system of claim 1, wherein the cartridge further comprises:
a sample receiving pad, and wherein the at least one lateral flow assay test strip comprises a pair of lateral flow assay test strips, each lateral flow assay test strip being in contact with the sample pad such that when the fluid sample is received onto the sample pad, the fluid sample wicks onto each of the lateral flow assay test strips such that parallel reactions occur in the pair of lateral flow assay test strips.

8. The system of claim 1, wherein the lateral flow assay test strip further comprises:
a conjugate disposed in a conjugate zone upstream of the specific binding assay zone, the conjugate reacting in the presence of a first of a plurality of analytes to form the detectable response in the specific binding assay zone on the transport matrix.

9. The system of claim 8, wherein the conjugate is configured for binding HbA1c.

10. The system of claim 8, wherein the specific binding assay zone is located upstream of the general chemical assay zone, wherein the lateral flow assay test strip further comprises:
a conjugate removal zone between the specific binding assay zone and the general chemical assay zone.

11. The system of claim 10, wherein the conjugate removal zone is formed by adsorption of anti-conjugate antibodies.

12. The system of claim 10 wherein the conjugate removal zone is formed by impregnation with a material that binds to and immobilizes the conjugate.

13. The system of claim 12, wherein the conjugate binding material is an antibody directed against the conjugate.

14. The system of claim 12, wherein the conjugate binding material is a polymer capable of bridging between and immobilizing conjugate microparticles.

15. The system of claim 8, wherein the general chemical assay zone is located upstream of the specific binding assay zone.

16. The system of claim 15, wherein there is no conjugate removal zone between the general chemical assay zone and the specific binding assay zone.

17. The system of claim 15, wherein the conjugate zone is disposed between the general chemical assay zone and the specific binding assay zone.

18. The system of claim 8, wherein the conjugate comprises:
a labeled indicator reagent diffusively immobilized on the transport matrix.

19. The system of claim 18, wherein the labeled indicator reagent comprises colored microparticles.

20. The system of claim 18, wherein the labeled indicator reagent comprises fluorescent microparticles.

21. The system of claim 8, wherein the labeled indicator reagent is a colored microparticle conjugated to an anti-HbA1c antibody.

22. The system of claim 18, wherein the first analyte is an HbA1c antigen.

23. The system of claim 18, wherein the labeled indicator reagent is a particle conjugated to a specific binding partner of the first analyte.

24. The system of claim 18, wherein the labeled indicator reagent is a particle conjugated to an analyte or analog of the first analyte.

25. The system of claim 18, wherein the labeled indicator reagent reacts in the presence of the first analyte to form a mixture containing a first analyte:labeled indicator complex.

26. The system of claim 8, further comprising:
a chemical indicator deposited upstream of the general chemical assay zone.

27. The system of claim 26, wherein the chemical indicator is configured to react chemically in the presence of a second analyte to form a detectable response in the general chemical assay zone on the transport matrix.

28. The system of claim 27, wherein the detectable response in the specific binding assay zone is formed from both the first and second analytes, and the detectable response in the general chemical assay zone is formed only from the second analyte.

29. The system of claim 26, wherein chemical indicator converts any hemoglobin present in the sample to met-hemoglobin.

30. The system of claim 1, wherein the specific binding assay is a competitive inhibition immunoassay.

31. The system of claim 1, wherein the specific binding assay is a direct competition immunoassay.

32. The system of claim 1, wherein the specific binding assay is a sandwich immunoassay.

33. The system of claim 1, wherein the general chemical assay uses a chemical indicator for direct colorimetry.

34. The system of claim 1, wherein the specific binding assay is used to detect the level of HbA1c in the sample, and the general chemical assay is used to detect the level of total hemoglobin present in the sample.

35. The system of claim 1, wherein the specific binding assay is used to detect the level of human albumin present in the sample, and the general chemical assay is used to detect the level of creatinine present in the sample.

36. The system of claim 1, wherein the measurement system is configured to determine the level of the selected analyte in the specific binding assay zone by comparison to the corresponding total detectable response in the general chemical assay zone.

37. The system of claim 1, wherein the logic circuit is configured to correct the level of the selected analyte in the specific binding assay zone by comparison to the corresponding detectable response in the general chemical assay zone.

38. The system of claim 1, wherein the logic circuit comprises:
pre-stored analyte calibration information.

39. The system of claim 38, wherein the logic circuit is configured to read the manufacturing lot identification information in the cartridge when the cartridge is received into the housing in order to confirm a proper match to the pre-stored calibration information.

40. The system of claim 1, wherein the body fluid analyte meter further comprises:
an autostart circuit configured to activate the meter when the body fluid sample is received into the at least one lateral flow test strip in the cartridge.

41. The system of claim 1, wherein,
the housing comprises a V-shaped stop for centering and aligning the cartridge, and wherein,
the cartridge comprises a V-shaped notch configured to be received against the V-shaped stop in the housing when the cartridge is received into the body fluid analyte meter.

42. The system of claim 1, wherein the housing has a fluid sample receiving opening, and the cartridge has a fluid sample receiving opening, and wherein the opening in the housing is disposed above the opening in the cartridge when the cartridge is received into the housing.

43. The system of claim 1, further comprising:
a sample preparation device configured to dispense the fluid sample into the opening in the cartridge.

44. The system of claim 1, further comprising:
a sample preparation device configured to dispense the fluid sample into the opening in the housing.

45. The system of claim 43, wherein the sample preparation device comprises a diluent.

46. The system of claim 43, wherein the sample preparation device comprises at least one of the group consisting of a surfactant, a buffer, and sodium ferricyanide.

47. The system of claim 1, wherein the transport matrix is in the form of an elongated strip having a proximate end containing the conjugate zone, a central section containing the specific binding assay zone and a distal end containing the general chemical assay zone.

48. The system of claim 1, wherein the transport matrix is in the form of a membrane stack with a first membrane containing the conjugate zone, a second membrane containing the general chemical assay zone and a third membrane containing the specific binding assay zone.

49. The system of claim 48, wherein the first membrane is positioned on top of the second membrane and the second membrane is positioned on top of the third membrane.

50. The system of claim 1, wherein the fluid sample is lysed whole blood.

51. The system of claim 1, wherein the transport matrix comprises a single continuous membrane made of the same material.

52. The system of claim 1, wherein the transport matrix comprises at least two membranes made of different materials in physical contact with each other.

53. The system of claim 52, wherein the at least two membranes are in end-to-end contact.

54. The system of claim 52, wherein the adjacent ends of the at least two membranes are overlapped.

55. The system of claim 52, wherein the at least two membranes are positioned one on top of the other.

56. The system of claim 52, wherein the conjugate zone and specific binding assay zone are located on a first membrane, and the general chemical assay zone is located on a second membrane.

57. The system of claim 52, wherein the first membrane is nitrocellulose, and wherein the second membrane is nylon.

58. The system of claim 52, wherein the conjugate zone is located on a first membrane, and the specific binding assay zone and the general chemical assay zone are located on a second membrane.

59. The system of claim 56, wherein the conjugate removal zone is formed by the junction between the first and second membranes.

60. The system of claim 8, wherein the transport matrix comprises at least two membranes made of different materials in physical contact with each other, and wherein the conjugate is disposed on a third membrane in contact with and upstream from the first membrane.

61. The system of claim 60, wherein the conjugate is disposed on the third membrane adjacent to the location where the first and third membranes contact one another.

62. The system of claim 61, wherein the conjugate is disposed as a sprayed-on stripe on the third membrane.

63. The system of claim 61, wherein the third membrane is cellulose acetate.

64. The system of claim 1, wherein the cartridge further comprises:
a sample absorbent pad in contact with a downstream end of the lateral flow assay test strip for absorbing excess fluid sample therefrom.

65. A cartridge for use with a body fluid analyte meter, the cartridge comprising:
(a) at least one lateral flow assay test strip, the lateral flow assay test strip comprising:
(i) a lateral flow transport matrix;
(ii) a specific binding assay zone on the transport matrix for receiving a fluid sample and performing a specific binding assay to produce a detectable response, and
(iii) a general chemical assay zone on the transport matrix for receiving the fluid sample and performing a general chemical assay to produce a detectable response;
wherein the cartridge is dimensioned to be receivable into a body fluid analyte meter such that a measurement system in the body fluid analyte meter is positioned to detect the responses in the specific binding assay zone and the general chemical assay zone in the lateral flow assay test strip.

66. The cartridge of claim 65, wherein the cartridge is a single-use disposable device.

67. The system of claim 65, wherein the cartridge further comprises:
a sample receiving pad, and wherein the at least one lateral flow assay test strip comprises a pair of lateral flow assay test strips, each lateral flow assay test strip being in contact with the sample pad such that when the fluid sample is received onto the sample pad, the fluid sample wicks onto each of the lateral flow assay test strips such that parallel reactions occur in the pair of lateral flow assay test strips.

68. The system of claim 65, wherein the lateral flow assay test strip further comprises:
a conjugate disposed in a conjugate zone upstream of the specific binding assay zone, the conjugate reacting in the presence of a first of a plurality of analytes to form the detectable response in the specific binding assay zone on the transport matrix.

69. The system of claim 68, wherein the conjugate is configured for binding HbA1c.

70. The system of claim 68, wherein the specific binding assay zone is located upstream of the general chemical assay zone, wherein the lateral flow assay test strip further comprises:
a conjugate removal zone between the specific binding assay zone and the general chemical assay zone.

71. The system of claim 70, wherein the conjugate removal zone is formed by adsorption of anti-conjugate antibodies.

72. The system of claim 70, wherein the conjugate removal zone is formed by impregnation with a material that binds to and immobilizes the conjugate.

73. The system of claim 72, wherein the conjugate binding material is an antibody directed against the conjugate.

74. The system of claim 72, wherein the conjugate binding material is a polymer capable of bridging between and immobilizing conjugate microparticles.

75. The system of claim 68, wherein the general chemical assay zone is located upstream of the specific binding assay zone.

76. The system of claim 75, wherein there is no conjugate removal zone between the general chemical assay zone and the specific binding assay zone.

77. The system of claim 75, wherein the conjugate zone is disposed between the general chemical assay zone and the specific binding assay zone.

78. The system of claim 68, wherein the conjugate comprises:
a labeled indicator reagent diffusively immobilized on the transport matrix.

79. The system of claim 78, wherein the labeled indicator reagent comprises colored microparticles.

80. The system of claim 78, wherein the labeled indicator reagent comprises fluorescent microparticles.

81. The system of claim 68, wherein the labeled indicator reagent is a colored microparticle conjugated to an anti-HbA1c antibody.

82. The system of claim 78, wherein the first analyte is an HbA1c antigen.

83. The system of claim 78, wherein the labeled indicator reagent is a particle conjugated to a specific binding partner of the first analyte.

84. The system of claim 78, wherein the labeled indicator reagent is a particle conjugated to an analyte or analog of the first analyte.

85. The system of claim 78, wherein the labeled indicator reagent reacts in the presence of the first analyte to form a mixture containing a first analyte:labeled indicator complex.

86. The system of claim 68, further comprising:
a chemical indicator deposited upstream of the general chemical assay zone.

87. The system of claim 86, wherein the chemical indicator is configured to react chemically in the presence of a second analyte to form a detectable response in the general chemical assay zone on the transport matrix.

88. The system of claim 87, wherein the detectable response in the specific binding assay zone is formed from both the first and second analytes, and the detectable response in the general chemical assay zone is formed only from the second analyte.

89. The system of claim 86, wherein chemical indicator converts any hemoglobin present in the sample to met-hemoglobin.

90. The system of claim 65, wherein the specific binding assay is a competitive inhibition immunoassay.

91. The system of claim 65, wherein the specific binding assay is a direct competition immunoassay.

92. The system of claim 65, wherein the specific binding assay is a sandwich immunoassay.

93. The system of claim 65, wherein the general chemical assay uses a chemical indicator for direct colorimetry.

94. The system of claim 65, wherein the specific binding assay is used to detect the level of HbA1c in the sample, and the general chemical assay is used to detect the level of total hemoglobin present in the sample.

95. The system of claim 65, wherein the specific binding assay is used to detect the level of human albumin present in the sample, and the general chemical assay is used to detect the level of creatinine present in the sample.

96. The system of claim 65, wherein the transport matrix is in the form of an elongated strip having a proximate end containing the conjugate zone, a central section containing the specific binding assay zone and a distal end containing the general chemical assay zone.

97. The system of claim 65, wherein the transport matrix is in the form of a membrane stack with a first membrane containing the conjugate zone, a second membrane containing the general chemical assay zone and a third membrane containing the specific binding assay zone.

98. The system of claim 97, wherein the first membrane is positioned on top of the second membrane and the second membrane is positioned on top of the third membrane.

99. The system of claim 65, wherein the fluid sample is lysed whole blood.

100. The system of claim 65, wherein the transport matrix comprises a single continuous membrane made of the same material.

101. The system of claim 65, wherein the transport matrix comprises at least two membranes made of different materials in physical contact with each other.

102. The system of claim 101, wherein the at least two membranes are in end-to-end contact.

103. The system of claim 101, wherein the adjacent ends of the at least two membranes are overlapped.

104. The system of claim 101, wherein the at least two membranes are positioned one on top of the other.

105. The system of claim 101, wherein the conjugate zone and specific binding assay zone are located on a first membrane, and the general chemical assay zone is located on a second membrane.

106. The system of claim 101, wherein the first membrane is nitrocellulose, and wherein the second membrane is nylon.

107. The system of claim 101, wherein the conjugate zone is located on a first membrane, and the specific binding assay zone and the general chemical assay zone are located on a second membrane.

108. The system of claim 105107, wherein the conjugate removal zone is formed by the junction between the first and second membranes.

109. The system of claim 68, wherein the transport matrix comprises at least two membranes made of different materials in physical contact with each other, and wherein the conjugate is disposed on a third membrane in contact with and upstream from the first membrane.

110. The system of claim 109, wherein the conjugate is disposed on the third membrane adjacent to the location where the first and third membranes contact one another.

111. The system of claim 110, wherein the conjugate is disposed as a sprayed-on stripe on the third membrane.

112. The system of claim 110, wherein the third membrane is cellulose acetate.

113. The system of claim 65, wherein the cartridge further comprises:
a sample absorbent pad in contact with a downstream end of the lateral flow assay test strip for absorbing excess fluid sample therefrom.

114. The cartridge of claim 65, wherein the cartridge further comprises:
an identification tag configured to be read by the meter.

115. The cartridge of claim 114, wherein the identification tag is an optically scanned barcode.

116. A lateral flow assay test strip, comprising:
(i) a transport matrix;
(ii) a specific binding assay zone on the transport matrix for receiving a fluid sample and performing a specific binding assay to produce a detectable response, and
(iii) a general chemical assay zone on the transport matrix for receiving the fluid sample and performing a general chemical assay to produce a detectable response, wherein the lateral flow assay test strip is formed from a single continuous membrane of material.

117. The lateral flow assay test strip of claim 116, wherein the specific binding assay zone is upstream of the general assay zone.

118. The test strip of claim 117, further comprising:
a conjugate removal zone disposed between the specific binding assay zone and the general chemical assay zone.

119. The test strip of claim 118, wherein the conjugate removal zone is formed by adsorption of anti-conjugate antibodies.

120. The test strip of claim 119, wherein the conjugate removal zone is formed by impregnation with a material that binds to and immobilizes the conjugate.

121. The test strip of claim 120, wherein the conjugate binding material is an antibody directed against the conjugate.

122. The test strip of claim 120, wherein the conjugate binding material is a polymer capable of bridging between and immobilizing conjugate microparticles.

123. The test strip of claim 116, wherein the specific binding assay zone is downstream of the general assay zone.

124. The test strip of claim 116, wherein the transport matrix is made of nitrocellulose.

125. The system of claim 116, wherein the lateral flow assay test strip further comprises:
a conjugate disposed in a conjugate zone upstream of the specific binding assay zone, the conjugate reacting in the presence of a first of a plurality of analytes to form the detectable response in the specific binding assay zone on the transport matrix.

126. The system of claim 125, wherein the conjugate is configured for binding HbA1c.

127. The system of claim 125, wherein the specific binding assay zone is located upstream of the general chemical assay zone, wherein the lateral flow assay test strip further comprises:
a conjugate removal zone between the specific binding assay zone and the general chemical assay zone.

128. The system of claim 127, wherein the conjugate removal zone is formed by adsorption of anti-conjugate antibodies.

129. The system of claim 127, wherein the conjugate removal zone is formed by impregnation with a material that binds to and immobilizes the conjugate.

130. The system of claim 129, wherein the conjugate binding material is an antibody directed against the conjugate.

131. The system of claim 129, wherein the conjugate binding material is a polymer capable of bridging between and immobilizing conjugate microparticles.

132. The system of claim 125, wherein the general chemical assay zone is located upstream of the specific binding assay zone.

133. The system of claim 132, wherein there is no conjugate removal zone between the general chemical assay zone and the specific binding assay zone.

134. The system of claim 132, wherein the conjugate zone is disposed between the general chemical assay zone and the specific binding assay zone.

135. The system of claim 125, wherein the conjugate comprises:
a labeled indicator reagent diffusively immobilized on the transport matrix.

136. The system of claim 135, wherein the labeled indicator reagent comprises colored microparticles.

137. The system of claim 135, wherein the labeled indicator reagent comprises fluorescent microparticles.

138. The system of claim 135425, wherein the labeled indicator reagent is a colored microparticle conjugated to an anti-HbA1c antibody.

139. The system of claim 135, wherein the first analyte is an HbA1c antigen.

140. The system of claim 135, wherein the labeled indicator reagent is a particle conjugated to a specific binding partner of the first analyte.

141. The system of claim 135, wherein the labeled indicator reagent is a particle conjugated to an analyte or analog of the first analyte.

142. The system of claim 135, wherein the labeled indicator reagent reacts in the presence of the first analyte to form a mixture containing a first analyte:labeled indicator complex.

143. The system of claim 125, further comprising:
a chemical indicator deposited upstream of the general chemical assay zone.

144. The system of claim 143, wherein the chemical indicator is configured to react chemically in the presence of a second analyte to form a detectable response in the general chemical assay zone on the transport matrix.

145. The system of claim 144, wherein the detectable response in the specific binding assay zone is formed from both the first and second analytes, and the detectable response in the general chemical assay zone is formed only from the second analyte.

146. The system of claim 143, wherein chemical indicator converts any hemoglobin present in the sample to met-hemoglobin.

147. The system of claim 116, wherein the specific binding assay is a competitive inhibition immunoassay.

148. The system of claim 116, wherein the specific binding assay is a direct competition immunoassay.

149. The system of claim 116, wherein the specific binding assay is a sandwich immunoassay.

150. The system of claim 116, wherein the general chemical assay uses a chemical indicator for direct colorimetry.

151. The system of claim 116, wherein the specific binding assay is used to detect the level of HbA1c in the sample, and the general chemical assay is used to detect the level of total hemoglobin present in the sample.

152. The system of claim 116, wherein the specific binding assay is used to detect the level of human albumin present in the sample, and the general chemical assay is used to detect the level of creatinine present in the sample.

153. A transverse flow assay test strip, comprising:
a transport matrix comprising a stack of membranes;
a specific binding assay zone on the transport matrix for receiving a fluid sample and performing a specific binding assay to produce a detectable response, and
a general chemical assay zone on the transport matrix for receiving the fluid sample and performing a general chemical assay to produce a detectable response.

154. The transverse flow assay test strip of claim 153, wherein the transport matrix comprises:
a membrane stack with a first membrane containing the conjugate zone, a second membrane containing the general chemical assay zone and a third membrane containing the specific binding assay zone.

155. The test strip of claim 154, wherein the first membrane is positioned on top of the second membrane and the second membrane is positioned on top of the third membrane.

156. The test strip of claim 155, wherein the detectable response in the general chemical zone is measurable from the membrane at the top of the stack and the detectable response in the specific binding assay zone is measurable from the membrane at the bottom of the stack.

157. The test strip of claim 153, wherein the detectable response in the general chemical zone is measurable from the membrane at the bottom of the stack and the detectable response in the specific binding assay zone is measurable from the membrane at the top of the stack.

158. A lateral flow assay test strip, comprising:
a lateral flow transport matrix;
a specific binding assay zone on the transport matrix for receiving a fluid sample and performing a specific binding assay to detect the level of human albumin present in the fluid sample, and
a general chemical assay zone on the transport matrix for receiving the fluid sample and performing a general chemical assay to detect the level of creatinine present in the fluid sample.
